(19) United States
(12) Patent Application Publication
(10) Pub. No.: US 2012/0078327 A1
(43) Pub. Date: Mar. 29, 2012

(54) MICRODEVICE-BASED ELECTRODE ASSEMBLIES AND ASSOCIATED NEURAL STIMULATION SYSTEMS, DEVICES, AND METHODS

(76) Inventors: Leif R. Sloan, Seattle, WA (US); Brad Fowler, Duvall, WA (US)

(21) Appl. No.: 13/308,931

(22) Filed: Dec. 1, 2011

Related U.S. Application Data
(63) Continuation of application No. 12/139,392, filed on Jun. 13, 2008.
(60) Provisional application No. 60/944,088, filed on Jun. 14, 2007.

(51) Int. Cl. A61N 1/375

(52) U.S. Cl. 607/72

(57) ABSTRACT

Microdevice-based electrode assemblies and associated neurostimulation systems, devices and methods are disclosed. A system in accordance with a particular embodiment includes a microdevice positioned to send signals or fluids to the patient, and/or to receive signals or fluids from the patient. The microdevice can include a housing having an external surface, and a signal/fluid transmitter/receiver positioned within the housing and coupled to a terminal carried by the housing. The system can further include a patient-implantable, flexible support member attached to the external surface of the housing and carrying the housing. The system can still further include an interface carried by the support member and connected to the terminal, with the interface being positioned to direct signals or fluids into patient tissue, and/or receive signals or fluids from the patient tissue.

### CROSS-REFERENCE TO RELATED APPLICATION

The present application is a continuation of U.S. application Ser. No. 12/139,392, filed Jun. 13, 2008, pending, which claims the benefit of U.S. Provisional Application No. 60/944,088, filed Jun. 14, 2007 and incorporated herein by reference.

### TECHNICAL FIELD

The present disclosure relates to systems, apparatus, devices, and methods that apply and/or receive signals and/or transfer substances in neural and/or other environments using structures, assemblies, and/or arrays that carry one or more microstimulators, microsensors, microinfusion pumps, and/or other types of devices.

### BACKGROUND

A wide variety of mental and physical processes are controlled or influenced by neural activity in particular regions of the brain. For example, the neural-functions in some areas of the brain (i.e., the sensory or motor cortices) are organized according to physical or cognitive functions. Several areas of the brain appear to have distinct functions in most individuals. In the majority of people, for example, the areas of the occipital lobes relate to vision, the regions of the left interior frontal lobes relate to language, and the regions of the cerebral cortex appear to be consistently involved with conscious awareness, memory, and intellect.

Many problems or abnormalities with body functions can be caused by damage, disease and/or disorders in the brain. Effectively treating such abnormalities may be very difficult. For example, a stroke is a common condition that damages the brain. Strokes are generally caused by emboli (e.g., vessel obstructions), hemorrhages (e.g., vessel ruptures), or thrombi (e.g., vessel clotting) in the vascular system of a specific region of the brain, which in turn generally cause a loss or impairment of a neural function (e.g., neural functions related to facial muscles, limbs, speech, etc.). Stroke patients are typically treated using various forms of physical therapy to rehabilitate the loss of function of a limb or another affected body part. Stroke patients may also be treated using physical therapy plus drug treatment. For most patients, however, such treatments are not sufficient, and little can be done to improve the function of an affected body part beyond the limited recovery that generally occurs naturally without intervention.

Neural activity can be influenced by electrical energy that is supplied from a waveform generator or other type of device. Various patient perceptions and/or neural functions can thus be promoted or disrupted by applying an electrical current to neural tissue. As a result, researchers have attempted to treat various neurological conditions using electrical stimulation signals to control or affect neural functions.

Some existing applications such as Transcranial Electrical Stimulation (TES), Deep Brain Stimulation (DBS), Vagal Nerve Stimulation (VNS), and Functional Electrical Stimulation (FES) attempt to treat particular neurological conditions using devices that provide electrical or magnetic energy to certain target locations. In such applications, electrodes are typically employed to deliver stimulation signals. The electrodes may be internal or external devices that are generally coupled to pulse generators by a set of wires.

For example, one existing technique involves implanting electrodes within a patient at a desired location for electrical stimulation, and implanting an implantable pulse generator (IPG) at a remote location. The IPG provides the stimulation signals and the electrodes deliver the signals. The IPG transfers signals to the electrodes by way of a set of lead wires that are tunneled through bodily tissues. Unfortunately, tunneling through tissue may be surgically invasive and/or difficult. Moreover, after implantation, bodily motion may stress portions of a tunneled lead wire, possibly adversely impacting system reliability.

In other forms of electrical stimulation, microstimulators may be employed to provide direct bipolar electrical stimulation to nerve or muscle tissues in an attempt to evoke a therapeutic response. The microstimulators are implanted at a target site by expulsion, such as through the lumen of a needle. FIG. 1A is a schematic illustration of an exemplary microstimulator known in the art, as disclosed in U.S. Pat. Nos. 5,193,539; 5,193,540; 5,324,316; and 5,412,367. FIG. 1B is a perspective illustration of another type of prior art microstimulator 80, as disclosed in U.S. Pat. No. 6,415,184.

With respect to FIG. 1A, the microstimulator 20 includes a capsule 40 in which electrical circuitry 44 and a power source 46 reside. The electrical circuitry 44 has a first and a second terminal 48a, 48b respectively coupled to a first and a second microstimulator electrode 42a, 42b by conductive wires. The capsule 40 is narrow and elongated, and hermetically seals the internal components of the microstimulator 20.

The miniature size of microstimulators may present certain difficulties in particular stimulation situations, possibly including migration from a target stimulation site over time and/or stimulation mode limitations. In light of such drawbacks, there is a need for a stimulation system and/or method that can provide simplified implantation procedures, enhanced reliability, and/or greater stimulation mode flexibility.

### BRIEF DESCRIPTION OF THE DRAWINGS

FIG. 1A is a diagrammatic illustration of a prior art microstimulator.

FIG. 1B is a diagrammatic illustration of another prior art microstimulator.

FIG. 2A is a schematic illustration of a Microdevice Based Electrode Assembly (MBEA) according to an embodiment of the disclosure.

FIGS. 2B, 2C, 2D, 2E and 2F are schematic illustrations of MBEAs according to other embodiments of the disclosure.

FIG. 2G is a schematic illustration of an implantable medical device that includes a conductive polymer material to facilitate electrical signal transfer.

FIG. 3 is a cross sectional schematic illustration of an MBEA according to an embodiment of the disclosure.

FIG. 4 is an exploded top isometric view of an MBEA according to an embodiment of the disclosure.

FIG. 5 is a cross-sectional illustration of an MBEA according to an embodiment of the disclosure.

FIG. 6 is a top isometric view of an electrical contact according to an embodiment of the disclosure.

FIGS. 7A, 7B, and 7C illustrate particular embodiments of MBEAs that include at least one microsensor configured and adapted for sensing or measuring one or more signals and/or substances in accordance with embodiments of the disclosure.

FIGS. 8A, 8B, and 8C are schematic views of MBEAs according to particular embodiments of the disclosure.

FIG. 9A is an illustration of a microstimulator based electrical stimulation system (MBESS) according to an embodiment of the disclosure.

FIG. 9B is a cross-sectional illustration of an MBEA implanted in a patient according to an embodiment of the disclosure.

FIG. 10A is a schematic illustration of an MBESS according to another embodiment of the disclosure.

FIG. 10B is a schematic illustration of a transcutaneous transmission patch.

FIG. 10C illustrates a schematic diagram of an MBESS according to another embodiment of the disclosure

FIG. 11 is an illustration of a microdevice based central-peripheral stimulation system (MBCPSS) according to an embodiment of the disclosure.

FIGS. 12A, 12B and 12C are top schematic views of MBEAs according to embodiments of the disclosure.

FIG. 13 is a cross section of an MBEA implanted in a patient and configured to provide unipolar stimulation according to an embodiment of the disclosure.

FIGS. 14 and 15 illustrate particular embodiments of remote electrodes.

FIGS. 16A and 16B are schematic illustrations of MBEAs according to other embodiments of the disclosure.

FIG. 17 is a schematic illustration of a microstimulation and microfluidic assembly in accordance with an embodiment of the disclosure.

### DETAILED DESCRIPTION

### Overview

The following disclosure describes various embodiments of systems, apparatus, devices, and methods in which one or more structures, assemblies, arrays, and/or members can be adapted and/or configured to carry one or more microstimulating elements, microsensing elements, microfluidic elements, signal transfer elements, and/or fluid transfer elements to facilitate the treatment, stimulation, monitoring, and/or evaluation of one or more target anatomical regions, tissues, sites, locations, and/or neural populations. The description herein details multiple embodiments of microdevice-based implantable assemblies (MBIAs), which in several embodiments comprise microdevice-based electrode assemblies (MBEAs), as elaborated upon below.

Various embodiments of the disclosure are directed toward the application or delivery of stimulation signals (e.g., electrical, magnetic, optical, acoustic, thermal, and/or other types of signals) to one or more neural populations. Such embodiments can include one or more types of microstimulators, as further detailed below.

In particular embodiments, a stimulation site is an anatomical region, location, or site at which such signals can be applied or delivered to, through, or near a target neural population. In various embodiments, one or more target neural populations reside within or upon one or more cortical regions, for example, a portion of the premotor cortex, the motor cortex, the supplementary motor cortex (SMA), the somatosensory cortex, the prefrontal cortex, and/or another cortical region. Additionally or alternatively, one or more target neural populations can reside elsewhere, for example, in a subcortical or deep brain region; within or upon the cerebellum; and/or upon or proximate to portions of the spinal cord and/or one or more cranial or other peripheral nerves.

A target neural population and/or a stimulation site can be identified and/or located in a variety of manners, for example, through one or more procedures involving anatomical landmark identification; electrophysiological signal measurement (e.g., electroencephalography (EEG) and/or electromyography (EMG)); neural imaging (e.g., Magnetic Resonance Imaging (MRI), functional MRI (fMRI), Diffusion Tensor Imaging (DTI), Perfusion Weighted Imaging (PWI), Positron Emission Tomography (PET), Near Infrared Spectroscopy (NIRS), Optical Tomography, Magnetoencephalography (MEG) and/or another technique); neurofunctional mapping (e.g., using Transcranial Magnetic Stimulation (TMS) and/or intraoperative stimulation); vascular imaging (e.g., Magnetic Resonance Angiography (MRA)); metabolite or chemical species spectrum analysis (e.g., Magnetic Resonance Spectroscopy (MRS)); and/or another type of functional, structural, and/or compositional anatomic assessment technique (e.g., Transcranial Doppler ultrasonography (TCD)). Representative techniques for identifying one or more target neural populations and/or stimulation sites are given in U.S. application Ser. No. 09/978,134 (published as US2004/0158298A1), which is incorporated herein by reference.

Some embodiments of the disclosure are additionally or alternatively directed toward the monitoring or sensing of electrical, thermal, chemical fluidic, and/or other states, properties, or activity associated with or corresponding to one or more neural populations and/or neurophysiologic or biological processes. Representative types of properties that can be relevant to particular embodiments include the presence, level, and/or absence of a drug, chemical marker, neurotransmitter, metabolite, bacterial or viral species, or other substance; blood oxygenation level; and cerebral blood flow (CBF) or cerebral blood volume (CBV). Such embodiments can include microsensor elements configured to monitor, detect, or sense particular types of activity or activity correlates at one or more monitoring sites.

A monitoring site in accordance with particular embodiments includes an anatomical region, location, or site at which signals and/or substances may be sensed or detected. Any given monitoring site can be identical to, essentially or generally identical to, associated with, or different from a stimulation site. A monitoring site can be identified in a variety of manners, for example, through or in association with one or more types of procedures described above (e.g., anatomical landmark identification, electrophysiological measurement, and/or medical imaging procedures).

Certain embodiments of the disclosure are additionally or alternatively directed toward the introduction, application, delivery, or release of one or more substances (e.g., a drug or a neurotrophic factor) to, through, or near particular tissues, which may include a neural population. Such embodiments can include particular types of microfluidic devices, as further described below. An infusion site can be defined as an anatomical region or location at which a substance can be introduced, applied, or delivered. An infusion site can be identified in several manners, for example, through an anatomical landmark identification procedure, a vascular imaging procedure (e.g., MRA), a neural imaging procedure (e.g., MRI or fMRI), a metabolite or chemical substance spectrum analysis procedure (e.g., MRS), and/or another type of procedure. An infusion site can be identical to, essentially or generally identical to, associated with, or different from a stimulation site.

Particular embodiments of the disclosure are additionally or alternatively directed toward the collection, removal, extraction or withdrawal of substances or fluids (e.g., cerebrospinal fluid (CSF)) from an anatomical location, and can include one or more microextraction devices to facilitate such withdrawal as further described below. An extraction site can include an anatomical location at or from which a desired type of substance may be collected, stored, and/or withdrawn, and can be identified through one or more types of procedures indicated above. An extraction site can be associated with or distinct from a stimulation or infusion site.

In the description that follows, particular embodiments that comprise one or more microstimulators can additionally or alternatively comprise one or more microsensors, microinfusion, and/or microextraction devices. Additionally, one or more sets of microdevices can be carried by a single structure or separate structures. Particular aspects of an embodiment described with reference to a microstimulator may equivalently or analogously apply to other embodiments involving other types of microdevices.

Various embodiments of the disclosure can perform stimulation, monitoring, infusion, and/or extraction operations in association with a treatment program that specifies or indicates one or more manners of treating, affecting, or influencing one or more types of neurologic dysfunction, functional deficits, conditions, and/or patient symptoms. A treatment program can provide for the application or performance of one or more treatments or therapies that are adjunctive or synergistic with respect to neural stimulation. An adjunctive or synergistic therapy can comprise, for example, a drug therapy, a neurotrophic and/or growth factor therapy, and/or a behavioral therapy. Depending upon embodiment details, a behavioral therapy can comprise a physical therapy activity, a movement and/or balance exercise, a strength training activity, an activity of daily living (ADL), a vision exercise, a reading task, a speech task, a memory or concentration task, a visualization, imagination, or role playing task, an auditory activity, an olfactory activity, a biofeedback activity, and/or another type of behavior, task, activity, or attempted activity that may be relevant to a patient's functional state, development, and/or recovery.

### Representative Electrode Assembly Structures

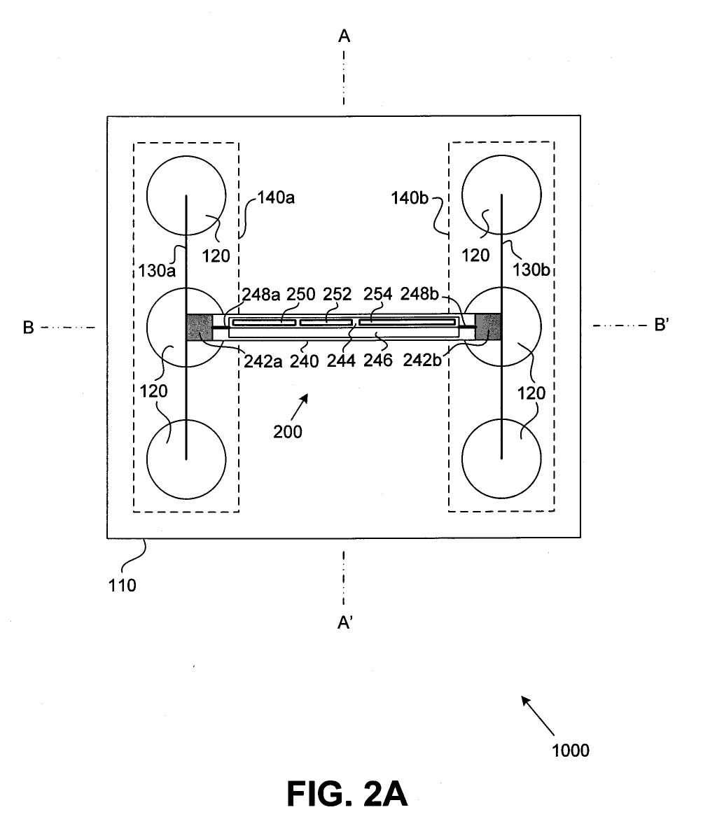

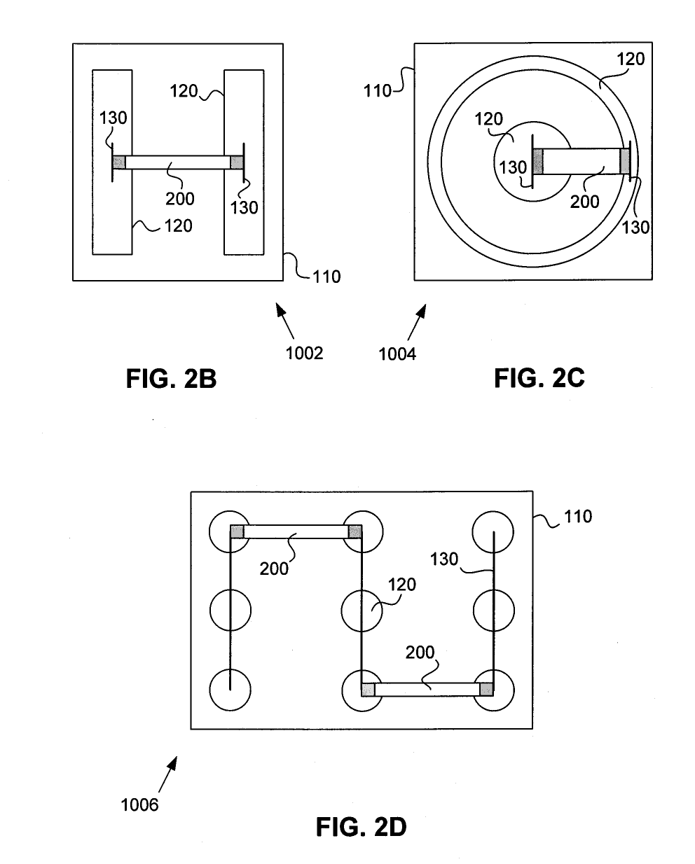

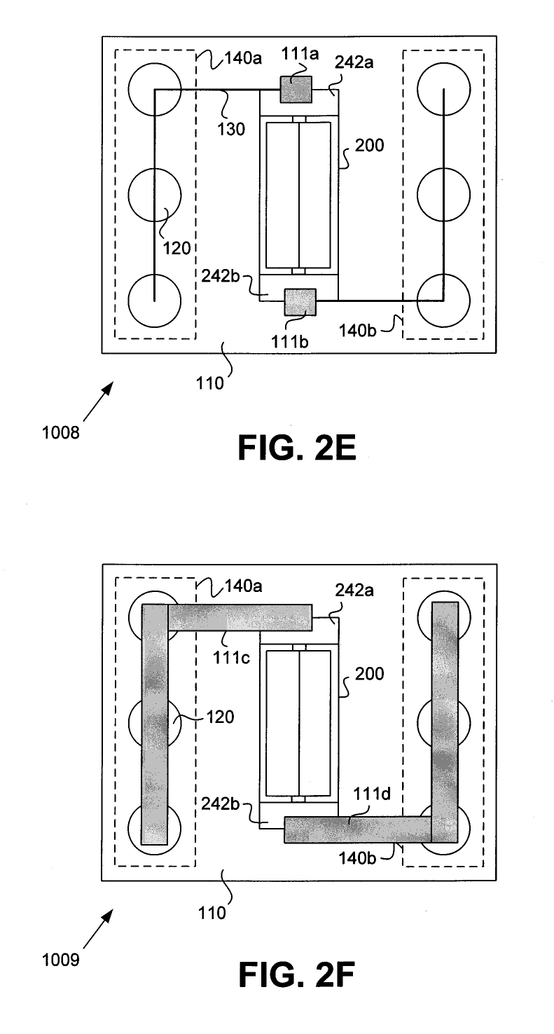

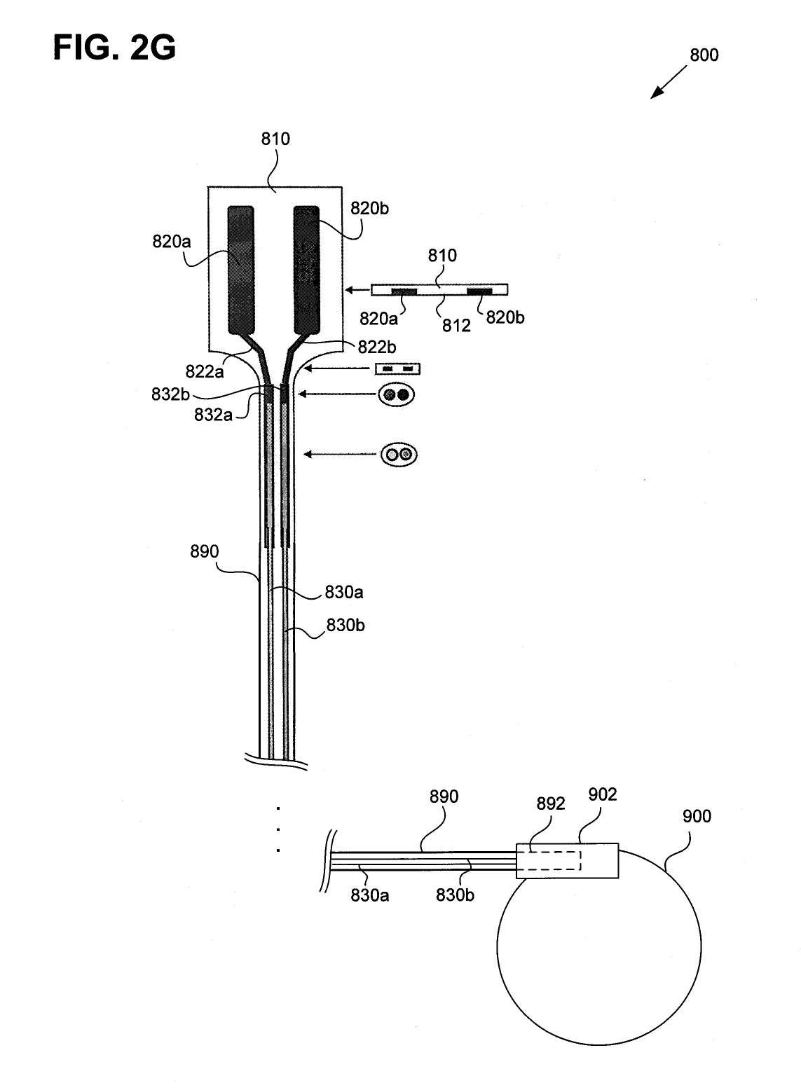

FIG. 2A is a plan view of a microdevice-based electrode assembly (MBEA) 1000 according to an embodiment of the disclosure. In various embodiments, an MBEA 1000 comprises at least one microstimulator 200, at least one support member 110, at least one signal transfer element such as a support member electrical contact or electrode 120, and possibly one or more other microdevices. In one embodiment, the microstimulator 200 and the electrical contacts or electrodes 120 are carried by the support member 110, and are electrically coupled by a set of conductive paths, conductive lines, links, and/or other conductive structures that can include but are not limited to lead wires. As further described below, one or more MBEAs 1000 carrying a set of microstimulators 200 and/or other types of microdevices can be remotely programmed, controlled, and/or interrogated by an external communication device, which communicates, for example, using RF, magnetic, optical, ultrasonic, and/or other types of signals. Particular microdevices can be configured to operate in an open loop manner or a closed loop manner relative to each other and/or an external communication device, controller, or computer. The term “microstimulator” is used herein to include small implantable microdevices that when used alone are typically positioned at or close to the target area (e.g., within several millimeters) and apply electrical signals to a patient, even if the benefit to the patient results from the inhibition of cells or activities at a target site. Accordingly, the microstimulator can provide excitatory, facilitatory and/or inhibitory signals, depending upon embodiment details. The signals provided by the microstimulator are referred to generally as “stimulation,” through they can have an excitatory, facilitatory and/or inhibitory effect, depending on embodiment details.

In certain embodiments, the electrical contacts 120 are organized as one or more sets or arrays. A first contact or electrode set 140a comprising one or more individual contacts 120 can be coupled to a first electrode 242a of the microstimulator 200, and a second contact or electrode set 140b comprising one or more individual contacts 120 can be coupled to a second electrode 242b of the microstimulator 200. A first and a second lead wire 130a, 130b can be electrically coupled to the microstimulator electrodes 242a, 242b and/or particular contacts 120 in a manner that reduces mechanical stress and/or enhances reliability, as further described below. The contacts or electrodes 120 can have a relatively large size when compared to the site of the microstimulator 200. For example, the electrodes 120 can have a diameter that is at least as large or larger than a diameter of the microstimulator 200.

FIG. 2A illustrates a 2×3 array of electrical contacts 120. Depending upon embodiment details, an MBEA 1000 can comprise additional or fewer contacts 120; contacts 120 exhibiting a different type of spatial arrangement; contacts 120 exhibiting different sizes and/or shapes; a microstimulator 200 exhibiting a different shape, size, and/or configuration; and/or more than one microstimulator 200. Representative examples of such MBEAs 1002, 1004, 1006 are schematically illustrated in FIGS. 2B, 2C, and 2D.

The support member 110 is comprised of a biocompatible material suitable for implantation within a patient. In various embodiments, the support member 110 is flexible, pliable, conformable, or at least generally flexible. In a representative embodiment, the support member 110 comprises a Silicone-based material. The shape of the support member 110 can vary, for example, depending on embodiment details, a particular neurological condition or application to which the MBEA 1000 is directed, and/or a stimulation site and/or target neural population under consideration. The size of the support member 110 can also vary, and in particular embodiments, the support member 110 is sized to be larger than the microstimulator 200. For example, the support member 110 can have a planform area (when viewed normal to its major surfaces) that is greater than the planform area of the microstimulator 200, e.g., at least twice as great in some embodiments, and at least five times as great in other embodiments. The support member 110 can also have a periphery that surrounds or encloses the periphery of the microstimulator 200. Both of the foregoing features, separately or together, can facilitate the ability of the support member 110 to carry the microstimulator 200. In some embodiments, the support member 110 can have a unitary (e.g., one-piece) construction, and/or can have the same general composition at the microstimulator 200 and at the electrical contacts 120. This arrangement can facilitate manufacturing in certain cases. In other embodiments described later, the composition of the support member can vary, e.g., to provide additional versatility. The support member 110 can be configured to facilitate ease of placement or positioning upon, near, or relative to particular neural locations. As used herein, “near” means at least reasonably close to a target neural population, including adjacent, proximate to, touching, or within neural tissue, so that the devices carried by the support member have an effect on the neural tissue. Although shown as having a rounded or tapered rectilinear configuration, the support member 110 can exhibit one or more other shapes, e.g., circular and/or having recessed and/or cropped edges. A contoured shape can facilitate support member positioning or placement and may enhance a manner in which the support member 110 conforms to a surface corresponding to a stimulation site.

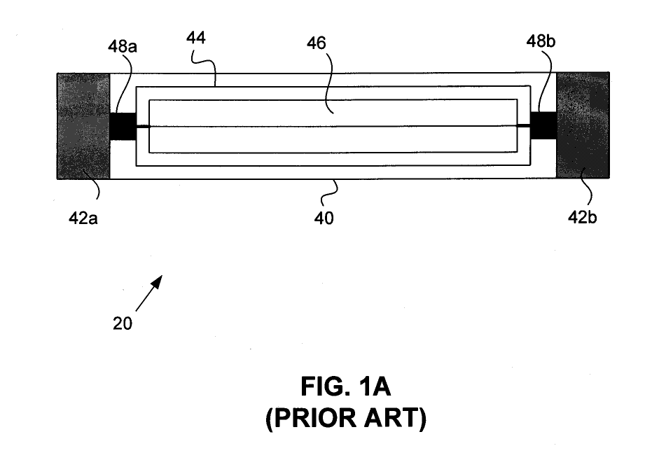

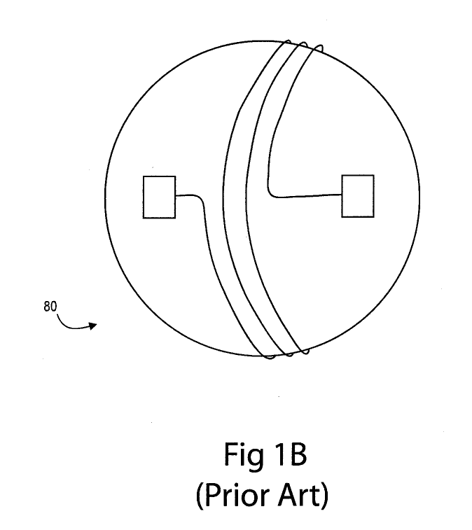

In a manner identical, essentially identical, analogous, or similar to that described above with reference to FIGS. 1A and/or 1B, in various embodiments a microstimulator 200 comprises a housing, capsule, or structure 240 in and/or upon which electrical circuitry 244 resides. The capsule or housing 240 can be hermetically sealed around the components inside, and has an external surface that is attached to the support member 110. Accordingly, the support member 110 does not extend into the interior space of the housing 240 in particular embodiments. The microstimulator 200 additionally comprises a power source 246. In one embodiment, a portion of the electrical circuitry 244 forms, provides, and/or is coupled to a first and a second internal terminal 248a, 248b within the capsule 240. The first and second terminals 248a, 248b are coupled to a set of microstimulator electrodes 242a, 242b that are accessible from outside the capsule 240 to facilitate signal transfer to and/or from signal transfer elements, electrical contacts 120, and/or tissues external to the capsule 240.

The capsule 240 can be comprised of, for instance, glass, ceramic, and/or other suitable materials that provide a hermetic package that excludes water vapor and/or bodily fluids but permits passage of signals (e.g., one or more types of power signals, configuration signals, commands and/or program instructions, and/or data signals). The electrodes 242a, 242b can be comprised of conductive materials such as, for example, tantalum and/or iridium, which may provide a biocompatible interface exhibiting minimal or negligible foreign-body reaction.

In general, the structure and function of the electrical circuitry 244 correspond to the capabilities that the microstimulator 200 provides or supports. In various embodiments, the electrical circuitry 244 comprises a control unit 250, a pulse unit 252, and a communication unit 254. The control unit 250 can comprise a processing unit, a state machine, and/or one or more other types of circuitry for directing microstimulator or microdevice operation. In various embodiments the control unit 250 also comprises one or more information storage elements (e.g., a register or a buffer) and/or a programmable or configurable medium for storing stimulation and/or monitoring information (e.g., stimulation parameters and/or sensed signal values), configuration information, control parameters, program instructions, and/or data. Depending upon the types of stimulation and/or monitoring capabilities the microstimulator 200 provides or supports, the control unit 250 can comprise a pulse generating unit, a sensing unit, a signal processing unit, and/or other elements (e.g., capacitors, resistors, coils, and/or other circuitry) that facilitate stimulation signal generation and the performance of particular types of operations or functions.

The pulse unit 252 comprises circuitry for generating direct current and/or alternating current stimulation signals, for example, in one or more manners described in U.S. patent application Ser. No. 11/182,713 (published as US2006-0015153A1), which is incorporated herein by reference. Such signals can be provided at one or more subthreshold and/or suprathreshold levels, where a threshold can be defined as a signal level that is expected to induce or evoke a patient response or a change in a measurable or monitorable patient state. Depending upon stimulation site and/or embodiment details, a signal applied at or above a threshold level can evoke a motor or sensory response; a cognitive response such as an increase or decrease in a reaction time; an emotional response such as a patient-reported change in mood (e.g., a sadness or anxiety level); or another type of response (e.g., a change or shift in an neuroelectric or hemodynamic signal or signal correlate).

The communication unit 254 comprises circuitry for sending and/or receiving power, control, programming, and/or data signals by inductive, radio-frequency (RF), optical, acoustic, and/or one or more other types of wireless signal transfer. In several embodiments, the communication circuitry includes a coil to facilitate telemetric signal transfer.

The power circuitry 246 comprises one or more suitable power sources that facilitate energy storage, conversion, transfer, and/or generation. The power circuitry 246 can comprise one or more devices such as, but not limited to, a battery, a rechargeable battery (e.g., a lithium ion power source), a capacitor, a supercapacitor, and/or the like. If a power source is replenishable or rechargeable, recharging is facilitated through, for example, the transfer of RF, optical, ultrasonic, thermal, and/or other types of energy to the microstimulator 200.

Depending upon embodiment details, a microstimulator 200 can have dimensions ranging from about 0.25-6.0 mm in diameter and about 1.0-40.0 mm in length. In one representative embodiment, a microstimulator 200 is approximately 1.0-2.0 mm in diameter and approximately 15 mm in length. In some embodiments, a microstimulator 200 comprises a Bionic Neuron or BION™ (Advanced Bionics Corporation, Sylmar, Calif.) of a type identical or similar to that described above with reference to FIG. 1A. In other embodiments, a microstimulator 200 comprises a ball-shaped stimulation device, as described above with reference to FIG. 1B.

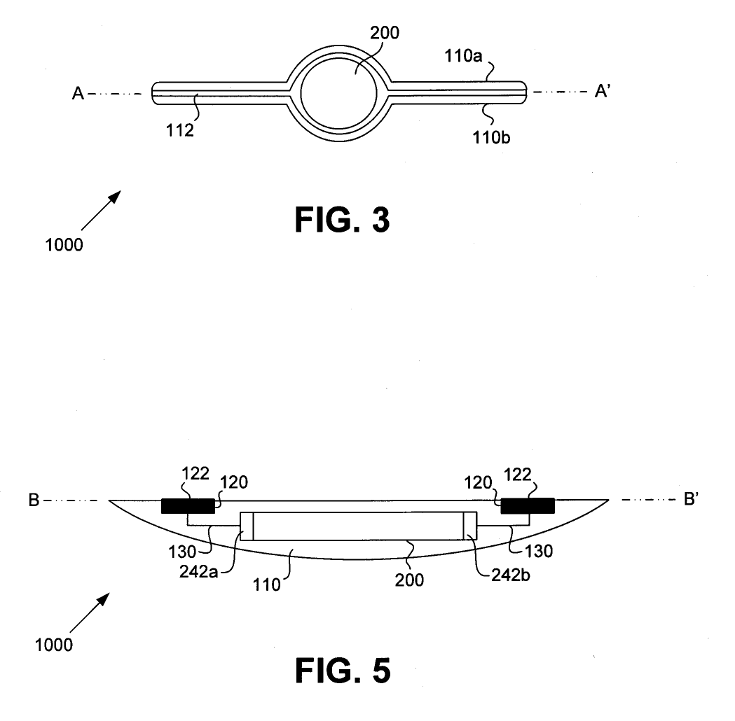

In certain embodiments, as illustrated in FIGS. 3-5, a microstimulator 200 is at least partially encapsulated, embedded within, surrounded by, and/or structurally coupled to portions of the support member 110. FIG. 3 is a cross sectional schematic illustration of an MBEA 1000 such as that shown in FIG. 2A along an axis A—A′. In particular embodiments, the support member 110 comprises a first layer 110a and a second layer 110b that can be sandwiched together using a suitable epoxy 112, thereby at least partially encapsulating the microstimulator 200. Some suitable epoxies include silicone elastomers (e.g., MED-4870, MED-6215 or MED-6755, manufactured by NuSil Technology of Carpinteria, Calif.). Each layer 110a, 110b can comprise one or more biologically compatible materials suitable for implantation, for example, a Silicone-based and/or other type of low durometer material. Low durometer materials provide flexibility and pliability, which can facilitate conformity to and/or placement at a stimulation and/or monitoring site.

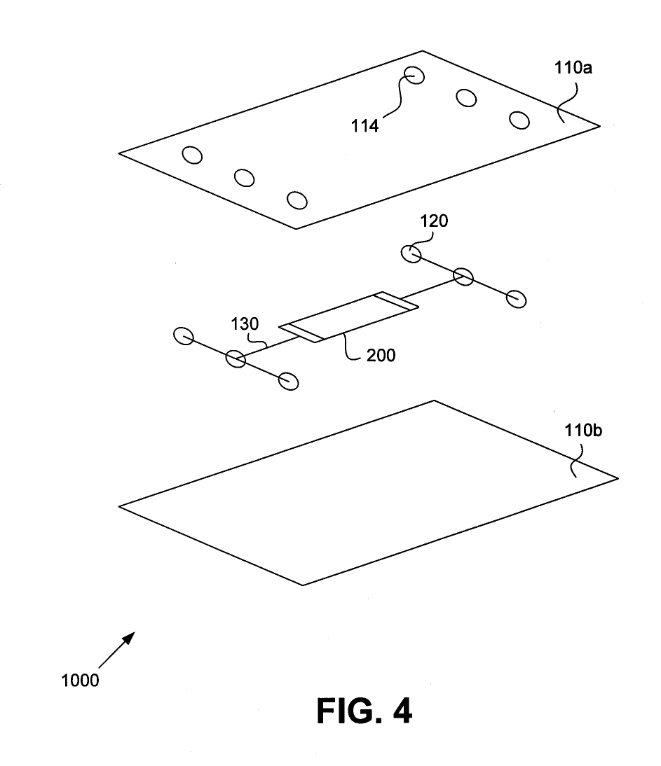

FIG. 4 is an exploded top isometric view of an MBEA 1000 such as that shown in FIGS. 2A and/or 3. Depending upon embodiment details, a support member's first layer 110a and/or second layer 110b can comprise one or more portions having identical, essentially identical, or different thicknesses. Essentially any surgically suitable bonding material can be used to join, bond, and/or fuse the contacts 120, the lead wires 130, and/or the microstimulator 200 to the first and second layers 110a, 110b in an essentially immovable, generally immovable, or motion limited manner.

In several embodiments, the first layer 110a includes a set of apertures 114 that facilitate signal transfer between particular contacts 120 and bodily tissue (e.g., a target neural population). In certain representative embodiments, an amount of surface area of a single electrical contact 120 that remains exposed or accessible through an aperture 114 is between approximately 0.25 mm and 10.0 mm. More particularly, in some embodiments an amount of exposed surface area per contact 120 is between approximately 0.5 mm and 6.0 mm, or approximately 1.0 mm and 5.0 mm. Depending upon embodiment details, one or more apertures 114 and/or contacts 120 can exhibit other dimensions. In any of these embodiments, the microstimlulator electrodes 242a, 242b (FIG. 2A) which are normally exposed to the patient for signal delivery, are instead connected to the lead wires 130 (or other conductive paths) at connection locations that are insulated, e.g., by the sandwich effect of the first and second layers 110a, 110b. The function performed by the now-insulated surfaces of the microstimulator electrodes 242a, 242b is instead provided by the support member electrodes or contacts 120.

FIG. 5 is a cross sectional schematic illustration of an MBEA 1000 such as that shown in FIG. 2A along an axis B-B′ showing a microstimulator 200 carried by a support member 110 according to an embodiment of the disclosure. The electrical contacts 120 are carried by the support member 110 in a manner that provides an exposed surface 122 for applying or delivering electrical signals to a stimulation site and/or receiving signals at a monitoring site. In embodiments such as that shown in FIG. 5, the microstimulator 200 is at least partially overmolded with the support member 110. An overmolded configuration provides an alternative to multiple support member layers described above. In this embodiment, a microstimulator 200 can be insert molded into a carrier material forming the support member 110. The carrier material can comprise essentially any material suitable for injection molding, such as a silicone elastomer. Particular processes for injection molding various elements of such an MBEA 1000 (which may include, for example, a microstimulator 200, lead wires 130 and/or electrical contacts 120) will be understood by those of ordinary skill in the art.

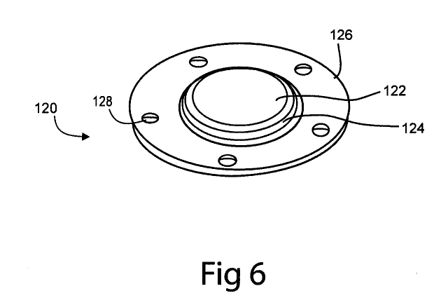

FIG. 6 is a top isometric view of an electrical contact 120 according to an embodiment of the disclosure. In one embodiment, a contact 120 includes a protruding or protracted surface or side 122, which can be defined as a tissue contact or communication side. A raised and/or sloping portion 124 forms a transition region between the protracted side 122 and a periphery 126. The periphery 126 includes a set of adhesive apertures 128 that facilitate bonding to the support member 110. In some embodiments, a groove, indentation, or recessed underside of the contact 120 can be coupled to a lead wire 130.

Depending upon embodiment details, one or more lead wires 130 and/or electrical contacts 120 can exhibit various shapes, sizes, and/or forms. For example, a contact 120 can have a raised portion 124 that is graduated or smoothly sloped, and although depicted as a substantially round shape with a circular contact surface, a contact 120 can take essentially any shape that is suitable for a desired type of stimulation and/or monitoring operation. Each lead wire 130 and/or contact 120 can comprise a biologically compatible electrically conductive material, for example, stainless steel, Gold, or Platinum-Iridium.

Representative types of support members, electrical contacts, and/or lead wire materials that are suitable for implementing various embodiments of the present disclosure are described in U.S. patent application Ser. No. 10/877,830 (published as US2005-0021118A1), incorporated herein by reference.

Referring again to FIG. 5, a lead wire 130 can be electrically and/or mechanically coupled to a microstimulator electrode 242a, 242b in various manners, for example, by a welding (e.g., laser welding), soldering, and/or annealing process; and/or a stamping, crimping, or mechanical pressurization process. In some embodiments, one or more portions of a microstimulator electrode 242a, 242b are formed, cast, molded, cut, stamped, and/or machined (e.g., with a notch or groove) to facilitate electrical coupling to a lead wire 130 having predetermined types of structural and dimensional characteristics (e.g., a known lead wire material composition, tensile strength, compression strength, thickness, length, and/or curvature). In certain embodiments, this occurs during a portion of a microstimulator manufacturing process, such that the microstimulator 200 itself and a set of lead wires 130 form a single or unified microstimulator/lead wire assembly in which a microstimulator electrode 242a, 242b can include one or more lead wires 130 integrally carried by and extending away from the microstimulator electrode 242a, 242b.

In addition to or as an alternative to the foregoing, to facilitate the formation of an electrically conductive interface between particular microstimulator electrodes 242a, 242b and particular lead wires 130, one or more portions or regions of a support member 110 that carry microstimulator electrodes 242a, 242b and lead wires 130 can include or be formed using a conductive polymer, such as a conductive Silicone, or a polymer of a type described in U.S. Patent Application Publication No. 20070060815. Such conductive support member portions can reside on or be formed in, for example, one or more inner support member surfaces that are electrically isolated from other portions of the support member 110 (e.g., external tissue contact surfaces of the support member, and other inner support member surfaces that are intended to be insulating or electrically isolated). Accordingly, the conductive polymer can form part of one or more conductive paths between the microstimulator electrodes 242a, 242b and associated support member electrodes 120. FIG. 2E is a top schematic view illustrating an embodiment of an MBEA 1008 having a support member 110 that carries a microstimulator 200 coupled to a set of electrical contacts by lead wires 130, where the support member 110 includes one or more conductive polymer coupling zones, regions, channels, paths, surfaces, or areas 111a, 111b that facilitate signal transfer between a microstimulator electrode 242a, 242b and a lead wire 130. In some embodiments, such as that shown in FIG. 2F, one or more lead wires 130 (FIG. 2E) themselves can be largely or entirely replaced by conductive polymer paths or channels 111c, 111d that are formed in and/or upon portions of the support member 110.

In general, one or more types of conductive polymers (and/or other types of conductive yet non-purely metallic materials) can be used to facilitate electromagnetic signal transfer in a variety of implantable medical systems and/or devices, whether such implanted medical systems or devices can include or omit microstimulators, microsensors, microfluidic devices, and/or other types of microdevices.

FIG. 2G is a schematic illustration of an implantable electrode assembly 800 that carries, includes, or incorporates a conductive polymer material to facilitate electromagnetic signal transfer between a signal source and a signal destination according to an embodiment of the disclosure. Also shown in FIG. 2G are representative cross-sectional views at several points along the length of the assembly 800. In various embodiments, the electrode assembly 800 includes a support member 810 that carries a set of electrical contacts 820a, 820b. At least one electrical contact 820a, 820b is coupled to a signal source such as an implantable pulse generator (IPG) 900 by way of a conductive pathway that includes a conductive polymer along at least a portion of its length.

Depending upon embodiment details, an electrical contact 820a, 820b can itself be partially or completely formed using a conductive polymer that is carried by a portion of the support member 810. In some embodiments, the support member 810 includes or is formed from an insulating Silicone-based (or other) material, and the electrical contacts 820a, 820b include or are formed from a conductive Silicone-based (or other) material. In particular embodiments, the support member 810 and electrical contacts 820a, 820b form a single type of polymeric material that exhibits spatial variations in its electrical conductivity profile (e.g., as a result of one or more molding, bonding, and/or doping processes). Any given electrical contact 820a, 820b can itself have a spatially varying conductivity profile or gradient, for example, along its length and/or width. The electrical contacts 820a, 820b can be essentially any type of shape and a variety of dimensions; for instance, one or more electrical contacts 820a, 820b can be circular, rectangular, annular, spiral, and/or another geometric configuration. In certain embodiments, one or more of the electrical contacts 820a, 820b can include metallic conductive elements, such as Platinum-Iridium or Gold, which can circular, rectangular, or otherwise shaped in a manner analogous to that for electrical contacts described above. Any given electrical contact 820a, 820b is carried by or located within the support member 810 such that it can apply an electrical signal to tissue at a tissue contact side 812 of the support member 810.

The support member 810 is structurally coupled to a lead body 890, which can accordingly form a portion of the support member 810. A set of a set of lead wires 830a, 830b at least partially extend along or through the length of the lead body 890. The lead wires 830a, 830b can be coupled to an IPG header 902 via a connector or terminal 892 to facilitate electrical signal transfer in a manner understood by those of ordinary skill in the art. Depending upon embodiment details, the lead wires 830a, 830b can be insulated (e.g., coated with an insulator) or uninsulated. In the illustrated arrangement, the overall support member 810 can accordingly include an elongated flexible lead body portion having the connector 892 at its proximal end and the electrodes or electrical contacts 820a, 820b at its distal end. This arrangement can be used with microstimulators or, as shown in 2G, with larger stimulators, including those typically implanted at a subclavicular location.

The lead body 890 includes a set of lumens, openings, channels, or pathways 832a, 832b along or through a portion of its length, where such lumens 832a, 832b carry or are at least partially filled with a conductive polymer such as conductive Silicone. Exposed or uninsulated portions of the lead wires 830a, 830b extend into and form electrical couplings with the conductive polymer within the lumens 832a, 832b. A conductive pathway or channel 822a, 822b electrically couples each lumen 832a, 832b to at least one electrical contact 820a, 820b, where the conductive channel 822a, 822b is carried by a portion of the support member 810 that forms a transition structure between the lead body 890 and other portions of the support member 810. Depending upon embodiment details, a conductive pathway 822a, 822b can be formed entirely or partially from a conductive polymer material.

In certain applications, it can be useful to sense, monitor, estimate, measure, evaluate, and/or characterize signals and/or substances at or near one or more stimulation and/or monitoring sites. As further described below, representative types of signals include electrical signals (e.g., a voltage or a current); physiological signals such as EEG, ECoG, and/or thermal signals; and/or physiological correlate signals such as coherence, cortical silent period, blood oxygenation level, and/or cerebral blood flow (CBF) information. In general, substances can correspond to particular types of fluids, biological species, chemical structures, chemical reactants, and/or reaction byproducts. Representative types of substances may include Cerebro-Spinal Fluid (CSF), drugs, neurotransmitters, hormones, growth factors, biological agonists and/or antagonists, and/or proteins.

The presence, absence, and/or characteristics of particular types of signals and/or substances can indicate whether a given type of condition or effect exists. Moreover, the presence, absence, and/or characteristics of particular signals and/or substances may provide an indication of when and/or how to apply a given type of therapy (e.g., neural stimulation, or an adjunctive therapy such as a behavioral and/or a drug therapy, which can be applied in association or conjunction with neural stimulation); when and/or how to adjust a therapy with respect to one or more time domains (e.g., a subseconds-based, a seconds-based, an hours-based, and/or other time domain); and/or when and/or how to initiate, interrupt, resume, or discontinue a therapy.

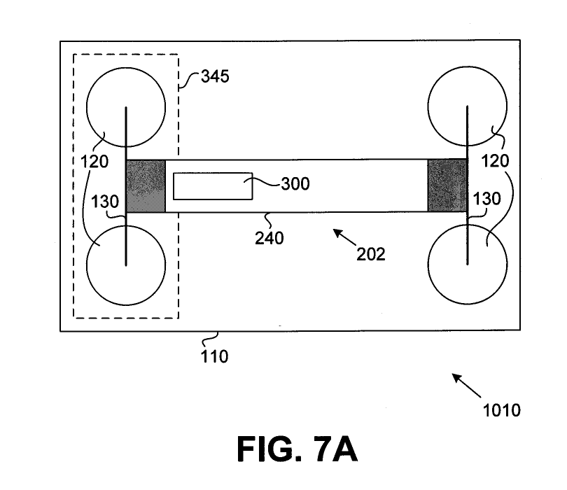

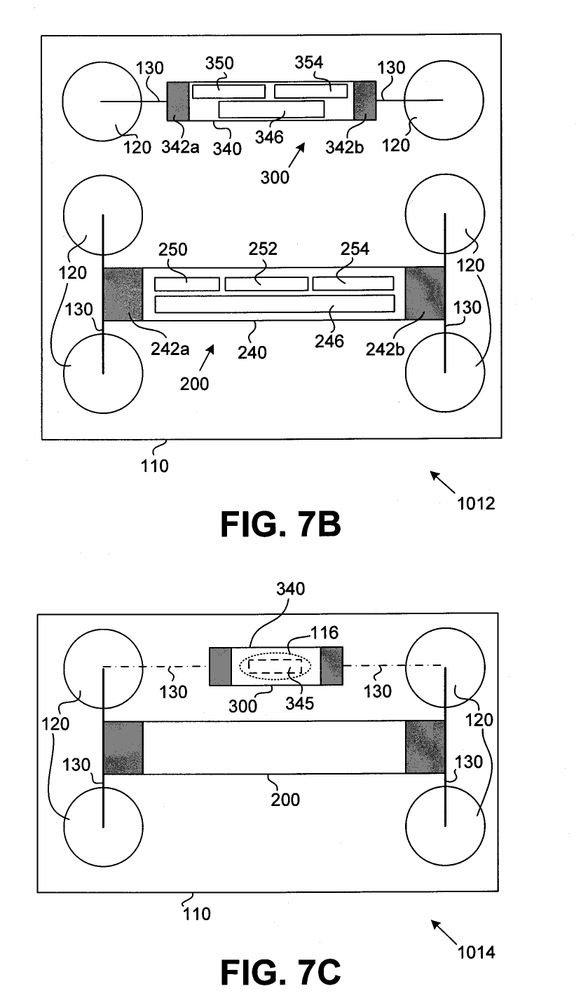

FIGS. 7A, 7B, and 7C illustrate particular embodiments of MBEAs 1010, 1012, 1014 that include at least one microsensor 300 configured and/or adapted for sensing or measuring one or more signals and/or substances. In some embodiments, a microstimulator 202 having a structure that is generally identical or similar to the microstimulator 200 described above with reference to FIG. 2A additionally comprises a microsensor 300 that is carried by, within, or adjacent to the microstimulator's capsule 240, for example, in a manner shown in FIG. 7A. Additionally or alternatively, as shown in FIGS. 7B and 7C, a microsensor 300 can be a separate device that is carried by a support member 110 and which resides external to a microstimulator 200. Other MBIA embodiments can include a support member 110 that carries one or more microsensors 300 without carrying a microstimulator 200, 202.

Depending upon embodiment details, a microsensor 300 can receive input in one or more manners. In various embodiments, a sensing interface 345 facilitates signal and/or substance transfer involving the microsensor 300. A sensing interface 345 can include, for example, certain electrical contacts 120, as shown in FIG. 7A; a set of openings or apertures or a permeable layer in a microsensor capsule 340, as depicted in FIG. 7C; and/or another structure. One or more portions of a support member 110 (e.g., an upper and/or a lower portion of a support member that provides an interface to bodily tissues and/or fluids) can include a set of sensing apertures 116 corresponding to a microsensor 300. A sensing interface 345 and/or a set of sensing apertures 116 through which a microsensor 300 detects signals and/or substances can reside upon the same and/or the opposite side of the support member 110 as particular electrical contacts 120 to which a microstimulator 200, 202 applies or delivers stimulation signals.

In some embodiments in which a microsensor 300 is an element of a microstimulator 202, and hence the microsensor 300 is carried by single housing or capsule 240, the microstimulator 202 and the microsensor 300 can have a shared control unit 250. Alternatively, the microsensor 300 and the microstimulator 200 can have separate control units 250, 350, as shown in FIG. 7B. Similarly, a microstimulator 200, 202 and a microsensor 300 can have shared or separate control units 250, 350, communication units 254, 354, and/or power sources 246, 346 depending upon embodiment details. In an embodiment shown in FIG. 7B, the microsensor unit also has a separate capsule or housing 340 and separate electrodes or other terminals 342a, 342b.

Particular contacts 120 can be dedicated or separately assigned to each of a microstimulator 200 and a microsensor 300; or a control unit 250 can manage signal transfer associated with contacts 120 shared between the microstimulator 200, 202 and the microsensor 300 in one or more manners (e.g., in response to specific commands, or in a predetermined, pseudo-random, or aperiodic time-multiplexed manner). In certain embodiments, a microsensor 300 can perform sensing or monitoring operations while a microstimulator 200, 202 outputs stimulation signals. In particular embodiments, the microsensor 300 includes signal filtering circuitry, signal subtraction circuitry, signal transformation circuitry, and/or other signal processing circuitry that facilitates the accurate identification of sensed signals relative to applied signals.

As previously indicated, a microsensor 300 can include one or more circuits, devices, and/or structures configured to sense or measure the presence, absence, and/or level of one or more types of signals, substances, parameters, and/or biological species, agents, analytes, or toxins at one or more times. A set of microsensors 300 can be configured to sense, for example, neuroelectric activity, tissue impedance, pH, tissue volume, and/or indirect indicators thereof such as blood flow, temperature, or pressure. One or more microsensors 300 can alternatively or additionally be configured to sense an oxygenation or deoxygenation level, a neurotransmitter level (e.g., dopamine, Gamma-aminobutyric acid (GABA), glutamate, epinephrine, norepinephrine, serotonin, and/or glycine); a hormone level; an enzyme level; a toxin level; a medication or drug level; and/or an infection or disease state marker.

The structure, characteristics, and/or capabilities of a microsensor 300 employed in any given embodiment may depend upon MBEA implantation site, an individual's physiological condition, and/or environmental factors. A microsensor 300 can include one or more types of sensing devices, for example, a chemically sensitive field-effect transistor (ChemFET). Depending on a substance being sensed, a ChemFET may comprise, for example, an Enzyme-Selective Field Effect Transistors (EnFET) and/or an Ion-Sensitive Field Effect Transistor (ISFET). A microsensor 300 can additionally or alternately include engineered substances (e.g., proteins, lipids, ion conduction channels, or chemical membranes), a set of chemically sensitive polymer layers, fluid capture or transfer elements, signal transfer or transducing elements, integrated circuit material layers or structures, nanostructures, optical elements (e.g., light emitting structures such as light emitting diodes (LEDs) or semiconductor lasers; optical fibers; and/or photodetectors), micromachined structures, and/or other elements.

As further described below, one or more microstimulators 200, 202 and/or microsensors 300 can be remotely programmed, interrogated, and/or activated by an external communication device. Particular microsensors 300 can be configured to operate in an open loop manner, or a closed loop manner in association with one or more microstimulators 200, 202, other microdevices, and/or an external communication and/or programming device.

### Enhanced Output and Switching Configurations

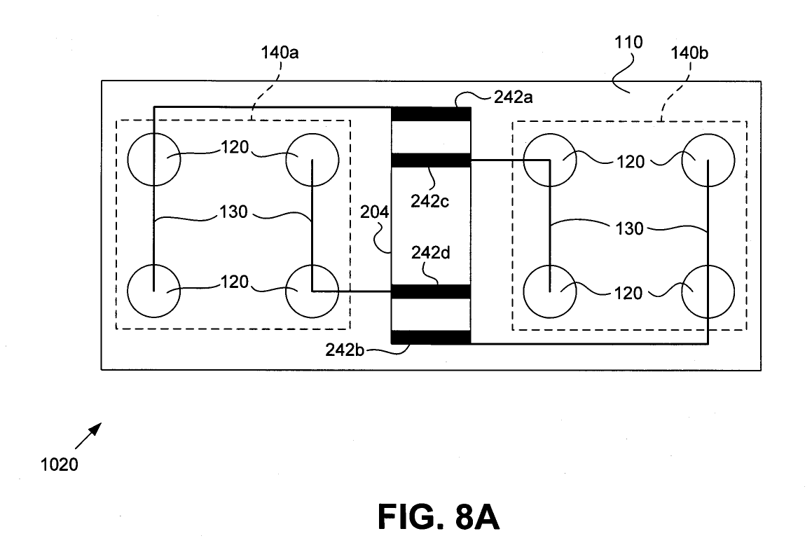

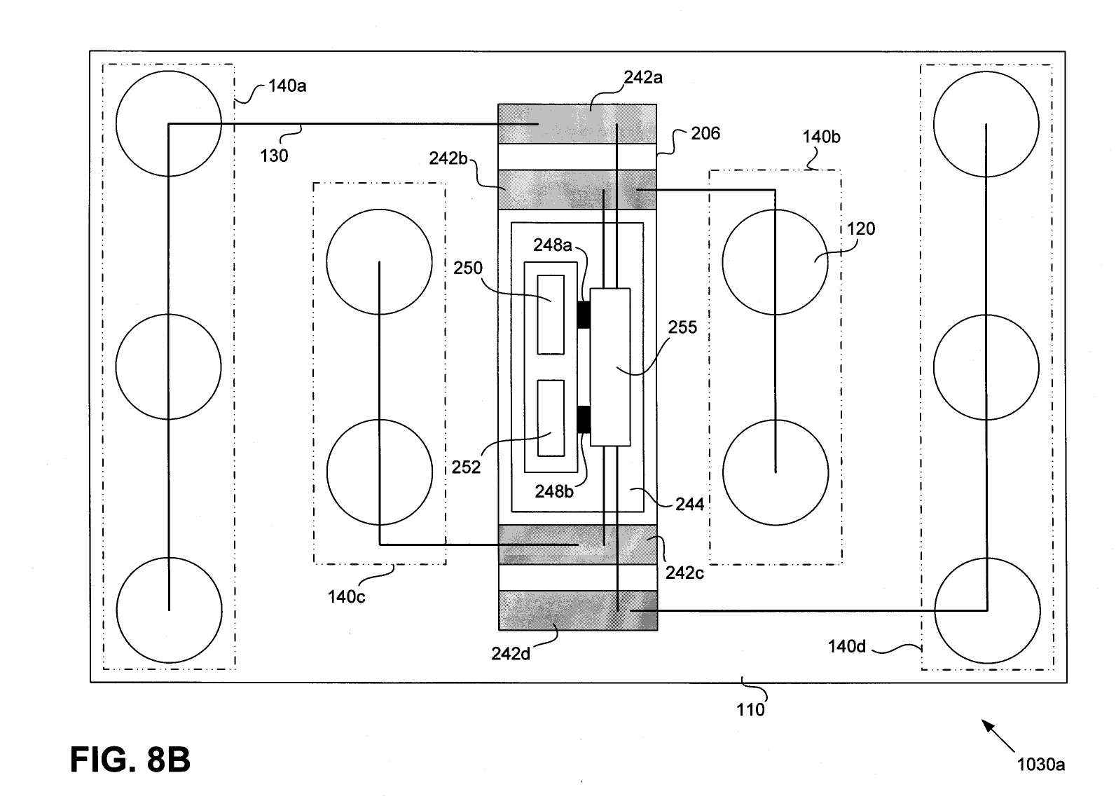

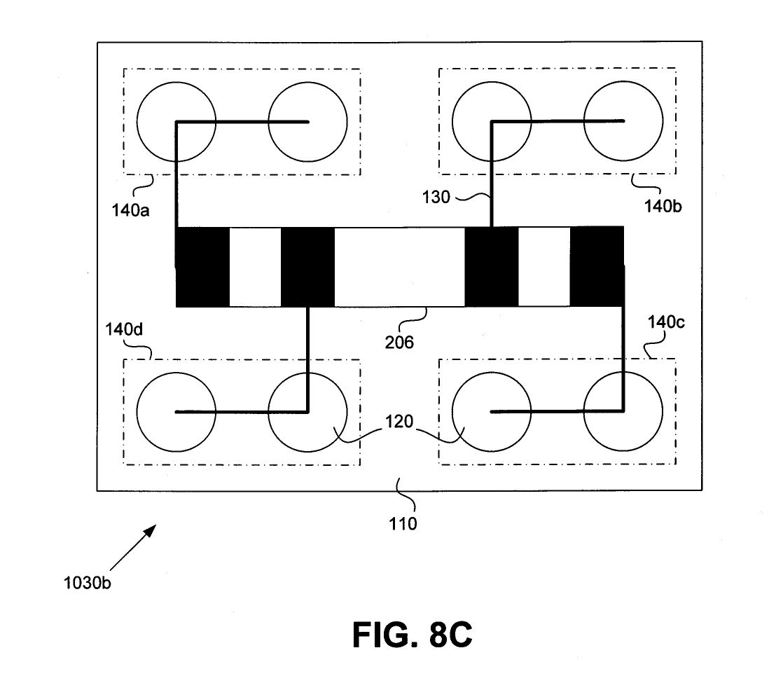

FIG. 8A is a top schematic view of an MBEA 1020 according to another embodiment of the disclosure. Relative to previously described Figures, like reference numbers may indicate like, similar, or analogous elements. The MBEA 1020 can include a support member 110, a set of electrical contacts 120, and at least one microstimulator 204 and/or other microdevice having more than two electrodes 242a-d. A set of lead wires 130 couple particular contacts 120 to particular microstimulator electrodes 242a-d, possibly in a manner that reduces mechanical stress and/or enhances reliability. Such lead wires 130 can be coupled to the microstimulator's electrodes 242a-d in a manner previously described.

An MBEA 1020 such as that shown in FIG. 8A can be designed and dimensioned for implantation relative to particular brain areas. For instance, the MBEA 1020 can be dimensioned such that in the majority of adult patients, one contact set 140a can deliver stimulation signals to portions of the cortex upon or proximate to the Sylvian fissure near the primary auditory cortex; and another contact set 140b can deliver stimulation signals to portions of the secondary auditory cortex and/or the secondary somatosensory cortex. Accordingly, a single device can be used to address disparate neurofunctional areas.

FIGS. 8B and 8C are top schematic views of MBEAs 1030a, 1030b according to another embodiment of the disclosure. Relative other Figures described herein, like reference numbers may indicate like, similar, or analogous elements. Referring first to FIG. 8B, in some embodiments, circuitry 244 within a microstimulator 206 includes a switching unit 255 that can be configured to couple the microstimulator's internal terminals 248a, 248b to particular microstimulator electrodes 242a-d at one or more times in a selectable or programmable manner. A microstimulator 206 can selectively establish a given coupling or signal routing pathway between its internal terminals 248a, 248b and its exterior or externally accessible microstimulator electrodes 242a-d in response to a command received from an external communication device and/or another implanted device (e.g., another microstimulator 200, 202, 204, 206, a microsensor 300, or other microdevice); and/or in association with a set of program instructions resident within the microstimulator 206 (e.g., within a buffer or computer readable medium), possibly in one or more of a predetermined, pseudo-random, or aperiodic manner with respect to one or more time domains. In a representative embodiment, the switching unit 255 includes a set of analog multiplexors, which can be controlled by the microstimulator's control unit 250.

Referring now to FIGS. 8B-8C, selective signal coupling and/or routing capabilities facilitate the application of stimulation signals to particular contacts 120 or contact sets 140a-d at one or more times, possibly in a manner that increases a likelihood of establishing, maintaining, restoring, and/or enhancing neural stimulation efficacy. For example, in association with a treatment optimization procedure, an MBEA 1030a, 1030b can direct stimulation signals to different contacts 120 to facilitate the identification of a neural population or subpopulation that most or least readily elicits a detectable patient response such as a sensation, a muscle movement, EMG activity, and/or another type of response. Moreover, such stimulation signal routing capabilities can facilitate stimulation site adjustment over time in response to changes in neural stimulation efficacy, treatment program parameters, and/or patient physiology.

Repeated, ongoing, or progressive variation of one or more stimulation signal parameters, possibly including a stimulation signal location and/or application pattern, can enhance neural stimulation efficacy and/or increase a likelihood of preventing or countering neural accommodation or habituation-like processes. An MBEA 1030a-b can apply stimulation signals to particular contact sets 140a-d in a manner that exhibits repeated, ongoing, or progressive variation with respect to one or more stimulation signal characteristics (e.g., current or voltage level, pulse repetition frequency, signal polarity, pulse phase widths, pulse burst patterns) and/or time domains in a programmable, predetermined, pseudo-random, and/or aperiodic manner.

For instance, within the context of a subseconds-based, a seconds-based, hours-based, and/or other activation time period, an MBEA 1030a-b can establish anode/cathode relationships between particular contacts 120 or one or more contact sets 140a, 140c while maintaining other contact sets 140b, 140d in an electrically inactive or floating state. Prior to or after the end of an activation time period currently under consideration, the MBEA 1030a-b can establish anode/cathode relationships between contacts 120 within one or more previously inactive contact sets 140b, 140d while at least one previously active contact set 140a, 140c remains in an active state or is switched to an inactive state. A given contact set 140a-d that is configured as an anode at one time can be configured as a cathode at another time. The duration of any given activation time period and/or a spatial activation pattern associated with any given contact set activation sequence can be determined in a predetermined, pseudo-random, or aperiodic manner (e.g., any activation time period may exhibit a predetermined, pseudo-random, or aperiodic relationship with respect to an integral multiple of a reciprocal of a pulse repetition frequency; and/or stimulation signals could be applied to contact sets 140a-d in a manner that defines or corresponds to a clockwise, counterclockwise, pseudo-random, and/or aperiodic pattern).

Those of ordinary skill in the relevant art will understand that in these and other MBEA embodiments, microstimulators 206 can have additional or fewer switched and/or non-switched electrodes 242a-d, and corresponding MBEAs 1030a-b can have additional or fewer contact groups 140a-d, one or more of which can include additional or fewer contacts 120 than shown in FIGS. 8B and 8C. In some embodiments in accordance with the present disclosure, a switched MBEA 1030a-b includes a microsensor 300 (FIGS. 7A-7C), which itself may include a switching unit to facilitate selective sensing operations.

### Representative Systems

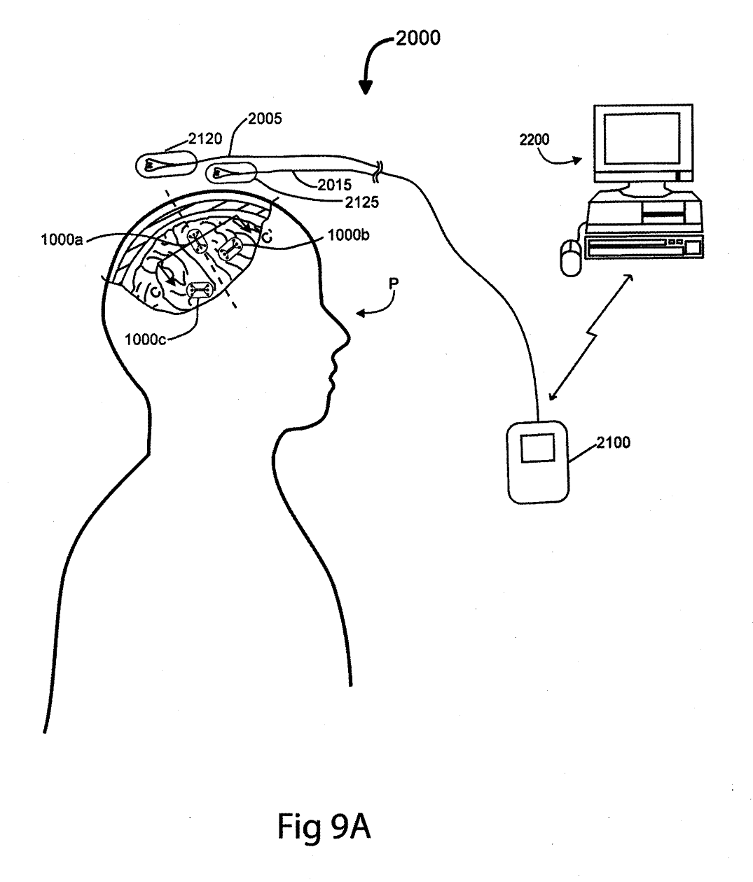

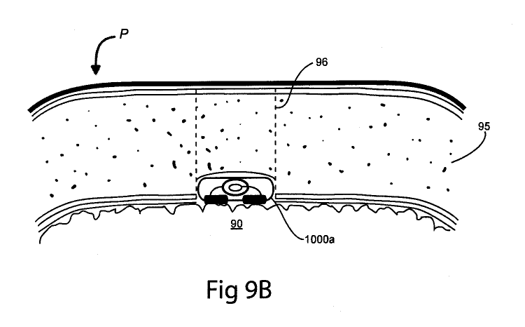

FIG. 9A is an illustration of a microstimulator based electrical stimulation system (MBESS) 2000 according to an embodiment of the disclosure. The MBESS 2000 comprises one or more MBEAs 1000a, 1000b, 1000c; an external programming device 2100; and one or more communication or signal transfer devices 2120, 2125. In general, an MBEA 1000 shown in FIG. 9A can be essentially any type of MBIA constructed in accordance with an embodiment of the disclosure.

An external programming device 2100 can include, for example, a control unit, a hand held programmer, a personal digital assistant (PDA), and/or a computer (not shown). A communication device 2120, 2125 further includes communication and control circuitry configured to facilitate signal transfer in accordance with a wireless signal transfer scheme (e.g., magnetic induction, RF signaling, infrared signaling, or any suitable scheme) known in the art. The programming device 2100 can transfer signals to and/or receive signals from each communication device 2120, 2125 by way of a corresponding wire-based or wireless link 2005, 2015. A given communication device 2120, 2125 can be positioned over, proximate to, or near one or more implanted MBEAs 1000a, 1000b, 1000c to facilitate wireless communication or signal transfer with particular microdevices.

In various embodiments, a set of MBEAs 1000a, 1000b, 1000c can be implanted and configured for neural stimulation and/or monitoring at spatially distinct locations. For example, a first MBEA 1000a can be configured to stimulate and/or monitor portions of the patient's motor cortex, premotor cortex, supplementary motor area (SMA), and/or somatosensory cortex; and a second MBEA 1000b can be configured to stimulate and/or monitor emotional and/or cognitive areas (e.g., portions of the prefrontal cortex, such as the dorsolateral prefrontal cortex (DLPFC), the ventrolateral prefrontal cortex (VLPFC), or the ventromedial prefrontal cortex (VMPFC), or the orbitofrontal cortex (OFC)); or a speech or language related region, such as a portion of Broca's area. Yet a third MBEA 1000c can be implanted relative to another brain area, for example, Wernicke's area, or a region associated with auditory processing such as a cortical location corresponding to the primary and/or secondary auditory cortex.

Spatially distinct neural stimulation and/or monitoring can involve MBEAs 1000a-c implanted relative to one or both brain hemispheres. Thus, a first MBEA 1000a can be configured to stimulate and/or monitor portions of the patient's cortex in one hemisphere, while another MBEA 1000b can be configured to stimulate and/or monitor homologous or nonhomologous portions of the patient's cortex in the other hemisphere. For instance, two MBEAs 1000 can be configured for bilateral stimulation of the motor cortex, or bilateral stimulation of cortical regions corresponding to and/or projecting into the auditory cortex.

In addition to the foregoing, one or more MBEAs 1000 can be implanted relative to other neural locations, for example, one or more locations along or proximate to the spinal column. Such MBEAs 1000 may be configured to perform stimulation and/or monitoring operations at one or more times. For example, a set of MBEAs 1000 can be implanted in a patient's back or neck proximate to the spinal column, and can apply stimulation signals and/or sense neural discharges corresponding to neural signaling volleys.

Based upon a type of neurologic state, condition, or dysfunction under consideration, stimulation of spatially distinct areas can occur in a simultaneous or sequential or interrupted manner. The stimulation can involve the use of identical and/or different stimulation parameters (e.g., stimulation amplitude, pulse width(s), pulse repetition frequency, burst frequency, stimulation modulation functions, electrical contact activation patterns, and/or other parameters) at one or more times.

Depending upon embodiment details, one or more MBEAs 1000 can be configured for stimulation and one or more MBEAs 1000 can be configured for sensing. A programming device 2100 can be configured to transmit power signals to some or all MBEAs 1000, and/or communicate information to and/or receive information from some or all MBEAs 1000. If an MBEA 1000 is configured for sensing, in certain embodiments, sensed information or signals corresponding thereto can be uploaded to the programming device 2100. Furthermore, in some embodiments, the programming device 2100 can be configured to transfer information to a computer system 2200, for example, for further processing of sensed signals. The computer system 2200 can include a processing unit and a computer readable medium for storage and/or processing of the received information. In some embodiments, the computer readable medium can be configured for storage, evaluation, and/or management of information relating to patient treatment history.

In some embodiments, a set of MBEAs 1000 can include one or more switching units (e.g., in a manner identical or analogous to that described above with respect to FIGS. 8A-8C), such that particular MBEAs 1000 can stimulate and/or sense distinct or generally distinct neurofunctional areas that are adjacent or proximate to each other simultaneously or at different times, possibly in a selectable, predetermined, random, and/or aperiodic manner. For instance, a single MBEA 1000 can be configured to apply stimulation signals to one set of contacts 140a-d (FIGS. 8A-8B) to stimulate a first cortical region corresponding to or having projections into the auditory cortex (e.g., the secondary auditory cortex); and apply stimulation signals to another set of contacts 140a-d to stimulate a second cortical region corresponding to the secondary somatosensory cortex. In an analogous manner, a given MBEA 1000 can stimulate a first cortical region corresponding to the motor cortex, and a second cortical region corresponding to the premotor cortex, the SMA, or the somatosensory cortex.

FIG. 9B is a cross sectional schematic illustration of an MBEA 1000a implanted in a patient P, taken along axis C-C′ shown in FIG. 9A. The MBEA 1000a is shown implanted within the patient's skull 95 and resting upon a neural surface. Although depicted in a general sense, it is to be appreciated that the MBEA 1000a can be located either above or upon a surface of the brain 90, including being located above, upon, and/or proximate to various structures such as the dura mater, pia mater, subdural space, arachnoid, subarachnoid space, and/or cortex. Implantation of the MBEA 1000a can involve a craniotomy, positioning the MBEA 1000a at a stimulation site, and insertion of a skull plug 96. In some embodiments, the skull plug 96 can be recessed, thinned, and/or contoured relative to adjacent or surrounding portions of the patient's skull 95 to accommodate an MBEA 1000a that exceeds a given thickness. This can reduce a likelihood of the MBEA 1000a applying an undesirable amount of force or pressure upon a neural surface when the skull plug 96 is inserted.

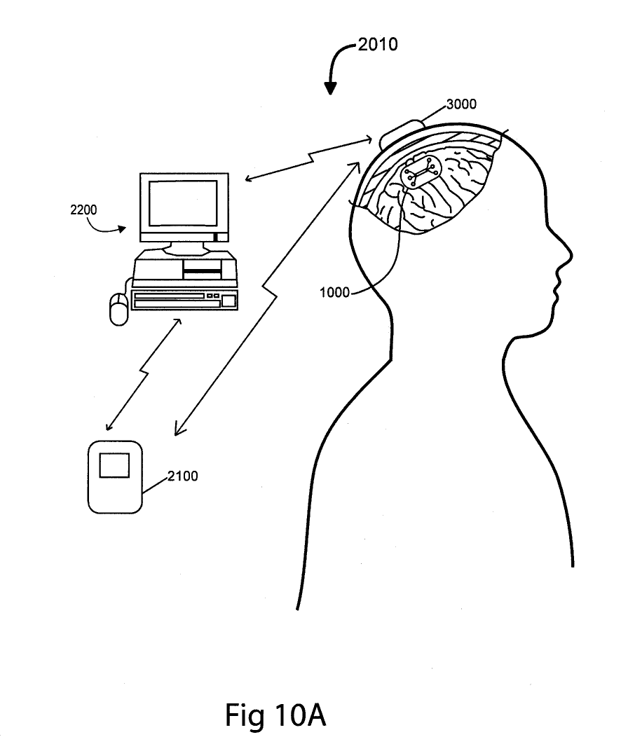

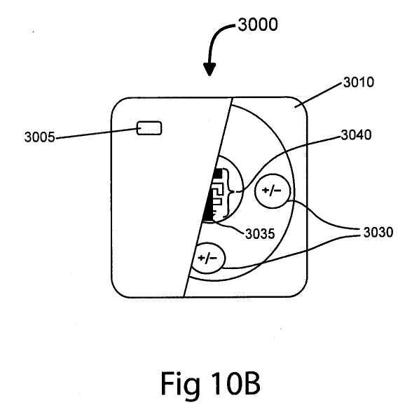

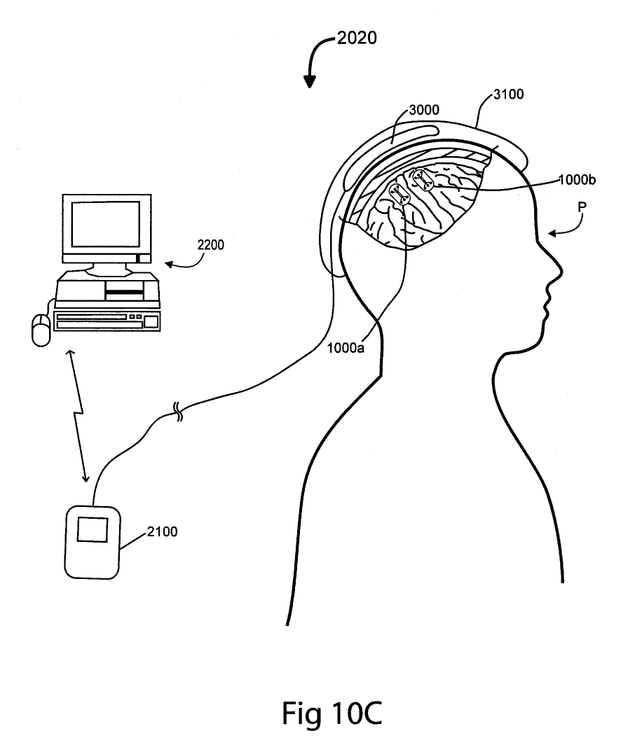

FIG. 10A is a schematic illustration of an MBESS 2010 according to another embodiment of the disclosure, in which a transcutaneous transmission patch (TTP) 3000 exchanges power and/or data signals with an implanted MBEA 1000. FIG. 10B is a schematic block diagram of a TTP 3000 with a top portion partially exposed or removed to show structural details. In various embodiments, the TTP 3000 comprises a flexible material forming a housing or platform 3010. The housing 3010 is adapted to carry an array of energy storage devices 3030 as well as a set of electronic components 3040. The electronic components 3040 can include circuitry such as one or more integrated circuits, an internal transmission coil, and/or other electronic circuitry. The TTP 3000 can transmit signals from a transmission coil 3035 through skin and other tissues to a receiving coil within one or more microdevices (e.g., microstimulators 200, 204, 206 described above). Energy storage devices 3030 can comprise replaceable and/or rechargeable power sources such as batteries, capacitors, supercapacitors, and/or the like. In some embodiments, the TTP 3000 additionally comprises a power actuator 3005 configured to selectively turn the TTP 3000 on or off. Additional embodiment details related to a similar or analogous transmission patch are disclosed in U.S. Pat. No. 5,948,006.

A TTP 3000 can be configured for one-way or two-way communication with an implanted MBEA 1000 (FIG. 10A). A TTP 3000 can additionally be configured to communicate with an external programming system, device or unit 2100 and/or computer system 2200. As a general example, for some therapies, a TTP 3000 transfers power signals to the MBEA 1000 to ensure sufficient power exists for proper operation for a given amount of time. A TTP 3000 can alternatively or additionally transfer commands or instructions to the MBEA 1000, for example, to establish or change stimulation parameters at one or more times. In another general example, it can be desirable for the TTP 3000 to obtain or receive data signals (e.g., sensed data) from the implanted MBEA 1000. In either example, signal transfer between the TTP 3000 and the MBEA 1000 is facilitated by TTP placement above, essentially above, or proximate to the MBEA 1000. In some embodiments, the TTP 3000 is adapted to be worn by a patient P by way of any suitable attachment structures and/or devices, which can comprise one or more of adhesives, headgear, straps, and/or fasteners configured for placement about the head of the patient P.

FIG. 10C illustrates a schematic diagram of an MBESS 2020 according to another embodiment of the disclosure, wherein a transmission cap 3100 that is worn upon the patient's head carries one or more signal transfer and/or communication systems, devices, and/or elements. In one embodiment, the transmission cap 3100 carries a TTP 3000, which may enhance the consistency or reliability of TTP positioning relative to one or more MBEA 1000a-b. In another embodiment, the transmission cap 3100 carries circuitry that is coupled to an external programming device 2100 or computer system (not shown) by a wire based or wireless link 2005. Such circuitry can include one or more transmission coils, power sources, and/or other components for communicating with an MBEA 1000 implanted in the patient P.

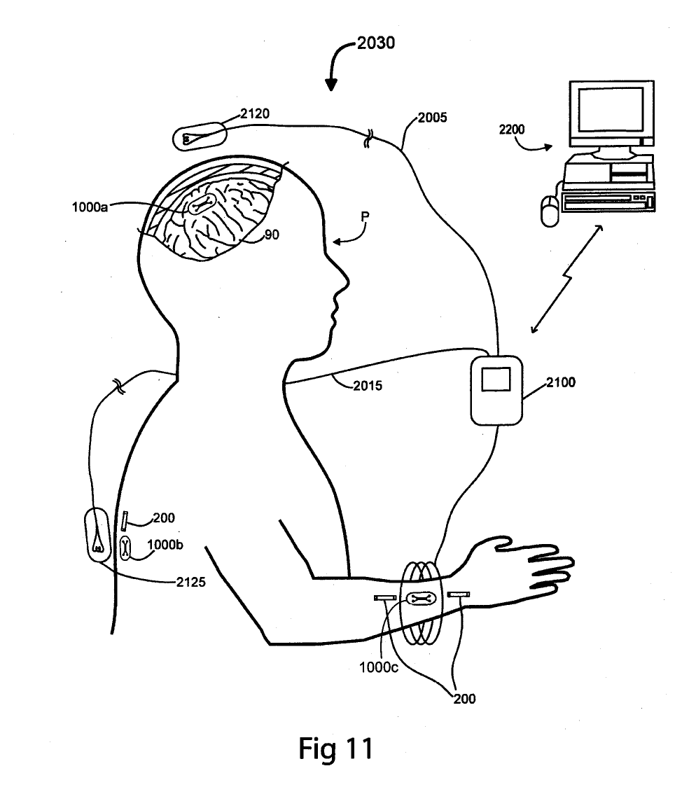

FIG. 11 is an illustration of a microdevice based central-peripheral stimulation system (MBCPSS) 2030 according to an embodiment of the disclosure. In various embodiments, the MBCPSS 2030 includes a set of devices configured to stimulate and/or monitor portions of the central nervous system (CNS), as well as a set of devices configured to stimulate and/or monitor portions of the peripheral nervous system (PNS). Depending upon embodiment details, a type of neurologic dysfunction under consideration, and/or the nature and/or extent of a patient's neurologic dysfunction, CNS stimulation and/or monitoring and PNS stimulation and/or monitoring can occur in a simultaneous, alternating, triggered, or independent manner. The timing of CNS stimulation and/or monitoring and PNS stimulation and/or monitoring relative to each other can be based upon or occur in accordance with an expected or actual signal conduction time associated with a neural pathway between central and peripheral nervous system regions. For example, subthreshold and/or suprathreshold brain and/or spinal column stimulation can be initiated, maintained, adjusted, interrupted, or discontinued based upon the detection of EMG activity associated with a portion of the PNS (e.g., an arm, wrist, or fingers). As another example, subthreshold and/or suprathreshold PNS stimulation can be initiated, maintained, adjusted, interrupted, or discontinued before or after CNS stimulation, or in response to the detection of neuroelectric CNS signals.

In one embodiment, the MBCPSS 2030 includes a set of devices configured to apply or deliver cortical stimulation, and a set of devices configured to apply or deliver functional electrical stimulation (FES). FES typically involves the application of electrical signals to one or more patient extremities, which can affect muscular movement and/or afferent neural signal transfer. In the embodiment shown in FIG. 11, the MBCPSS 2030 includes at least one MBEA 1000a implanted relative to a first cortical location and configured for stimulation and/or monitoring of one or more target neural populations within the brain 90; and a set of electrodes, MBEA 1000b-c, microstimulators 200, and/or other microdevices positioned or implanted relative to a second location, such as an arm, and configured for stimulation and/or monitoring of particular muscles and/or peripheral nerves. In a representative embodiment, an MBEA 1000a is implanted relative to a cortical location associated with sensory or motor processing of an affected body part, while one or more microstimulators 200, are implanted relative to or at one or more corresponding peripheral locations associated with the sensory or motor function.

As shown in FIG. 11, the MBCPSS 2030 can further include at least one external control and/or programming system, unit, or device 2100 for managing the operation of the implanted MBEAs 1000a-c and/or the peripherally implanted FES devices. In one embodiment, the control unit 2100 is coupled to a first 2120 and a second 2125 signal transfer or communication device by a first link 2005 and a second link 2015, respectively. The first and/or second links 2005, 2015 can be wire-based or wireless. The first and/or second communication devices 2120, 2125 comprise wireless signal transfer devices, for example, coils and associated circuitry that facilitate RF and/or another type of signal transfer and/or exchange. The control unit 2100 is configured to send signals (e.g., power signals; acknowledgement, handshaking, and/or security verification signals; commands; configuration information; instructions; and/or other signals) to and/or receive signals (e.g., data signals and/or other signals) from the centrally implanted MBEA 1000 and/or the peripherally implanted FES devices at one or more times.

### Unipolar Configurations

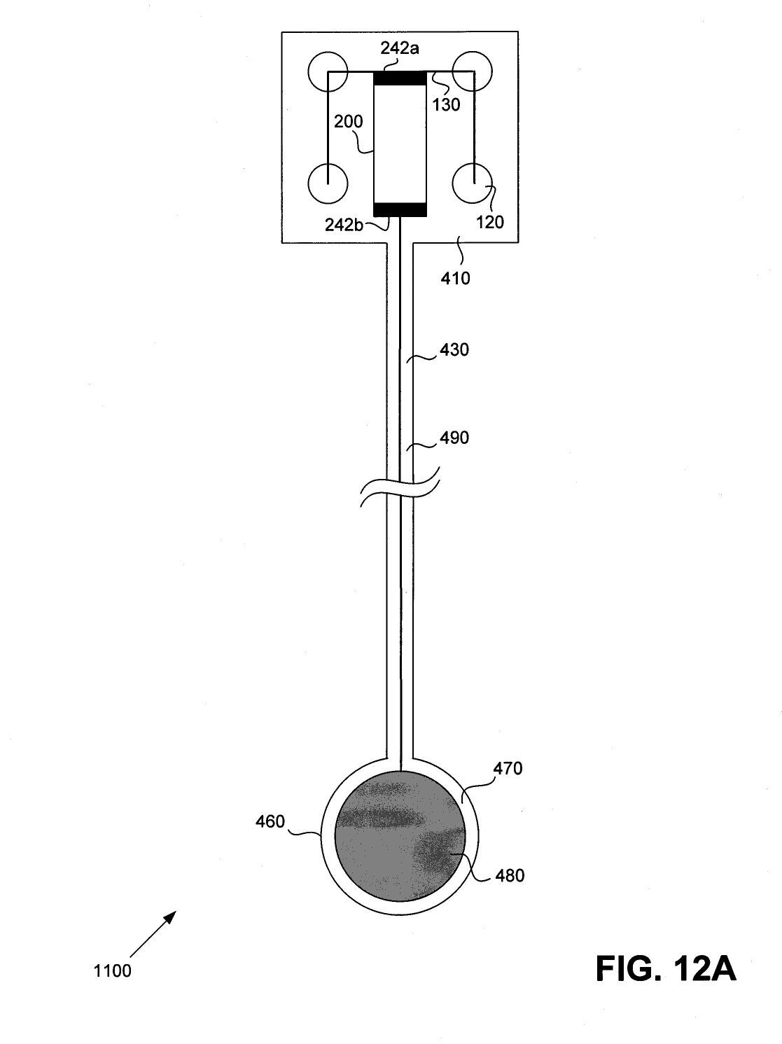

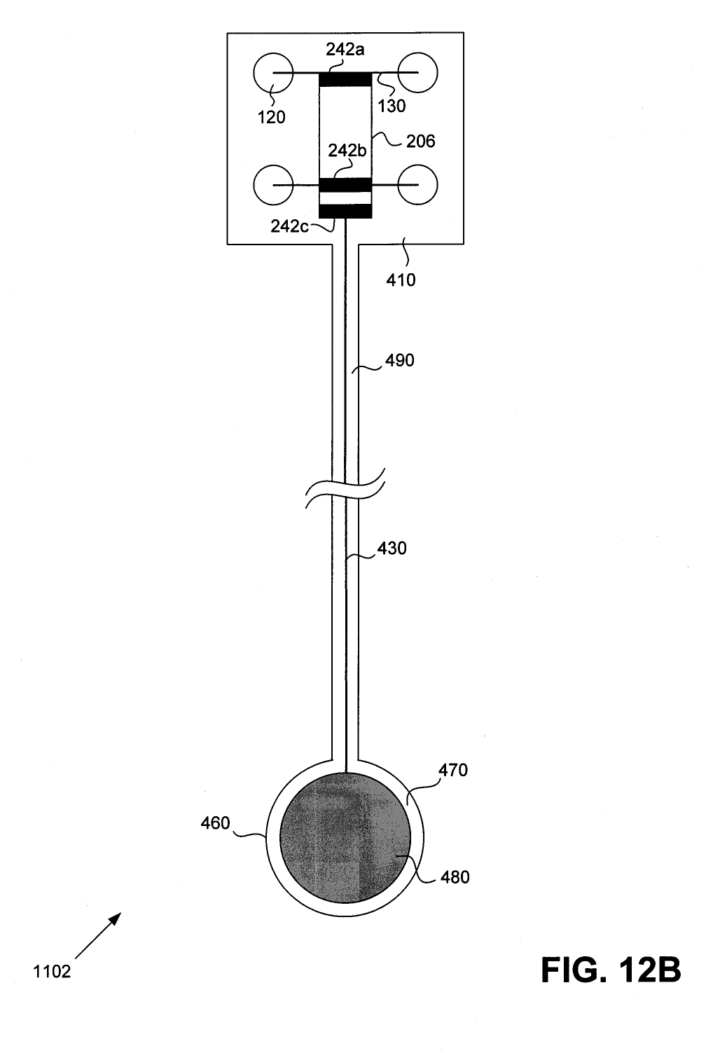

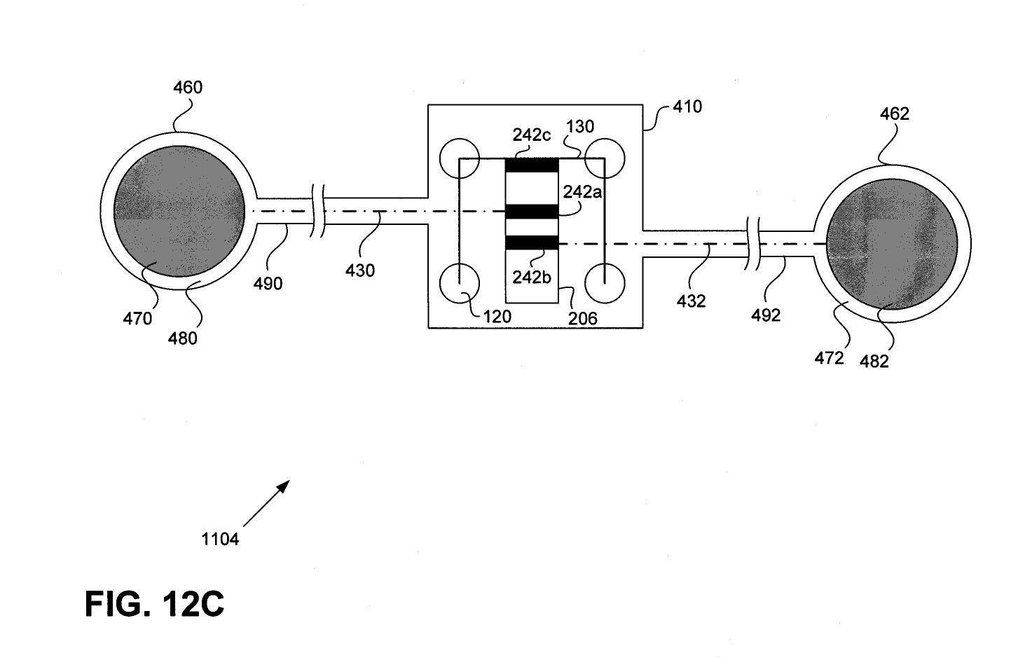

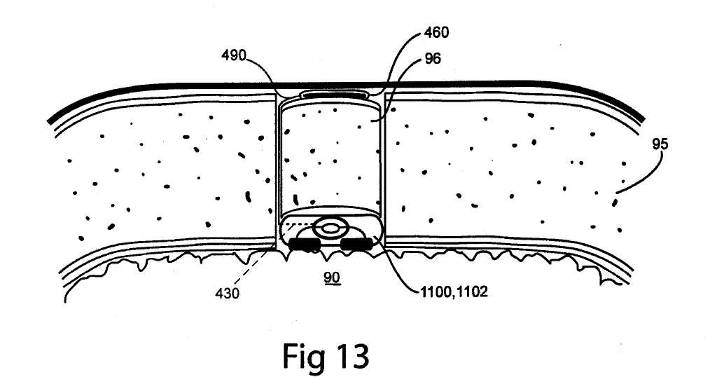

FIGS. 12A-13 illustrate various embodiments of MBEAs 1100, 1102, 1104 directed toward the application or delivery of identical polarity or unipolar stimulation signals to a stimulation site at one or more times. In a unipolar configuration, electrical continuity can be facilitated by positioning one or more electrodes, electrical contacts, and/or other signal transfer devices apart, distant, or remote from the stimulation site. For example, the signal transfer devices may be spaced apart by from about 1 to about 30 cm or more, and/or may be located adjacent to different neurofunctional populations. Accordingly, a current path in a unipolar configuration can span or include distant, separate, distinct, and/or generally distinct neurofunctional areas, bodily tissues, and/or anatomical locations. Unipolar stimulation can reduce power consumption, provide enhanced efficacy or efficiency stimulation, and/or mitigate collateral effects associated with stimulation. Relative to other Figures described herein, like or analogous reference numbers in FIGS. 12A-13 may indicate like or analogous elements.

FIGS. 12A and 12B are top schematic views of MBEAs 1100, 1102 according to embodiments of the disclosure. In one embodiment, an MBEA 1100 includes a first support member 410 that carries at least one electrical contact 120 and at least one microstimulator 200. The MBEA 1100 further includes a second support member 470 that carries at least one indifferent or remote electrical contact or signal transfer device 480 that facilitates electrical circuit completion. Taken together, the remote contact 480 and the second support member 470 comprise a remote electrode, electrode array, electrode structure, electrode assembly, or electrode device 460.

The microstimulator 200 can include a plurality of electrodes, for example, a first and a second electrode 242a, 242b as shown in FIG. 12A. The electrical contacts 120 carried by the MBEA's first support member 410 can be electrically coupled to the microstimulator's first electrode 242a by a first set of lead wires 130. The remote electrode 460 can be electrically coupled to the microstimulator's second electrode 242b by a return lead wire 430, which can be carried by a lead body 490. The lead body 490 can form a portion of the first and/or second support members 410, 470 in a contiguous or approximately contiguous manner. The lead body 490 can be a flexible or generally flexible biocompatible material (e.g., a Silicone-based material), in a manner understood by those of ordinary skill in the relevant art. In a unipolar configuration, electrical contacts 120 coupled to the microstimulator's first electrode 242a apply an identical polarity signal at any given time, while the remote electrode 460 is maintained at a neutral or opposite polarity to facilitate electrical circuit completion.

With respect to the MBEA 1102 shown in FIG. 12B, in one embodiment the microstimulator 206 includes a first, a second, and a third microstimulator electrode 242a, 242b, 242c. Particular contacts 120 can be coupled to the microstimulator's first and second electrodes 242a-b, and the remote electrode 460 can be coupled to the microstimulator's third electrode 242c. In an embodiment in which particular microstimulator electrodes 242a-c can be selectively activated or inactivated, for example, when the microstimulator 206 includes switching capabilities in a manner analogous to that described above, the MBEA 1102 can be selectively configured to provide bipolar stimulation as well as at least one type of unipolar stimulation. In a bipolar configuration, the first microstimulator electrode 242a is biased at a first polarity and the second microstimulator electrode 242b is biased at a second polarity that is defined to be neutral or opposite to the first polarity, while the third microstimulator electrode 242c remains inactive or electrically isolated. In a first unipolar configuration, the microstimulator's first and second electrodes 242a-b are biased at a first polarity, and the microstimulator's third electrode 242c is biased at a second, opposite polarity. In a second unipolar configuration, the microstimulator's first electrode 242a is biased at a first polarity, the microstimulator's second electrode 242b is held inactive or electrically isolated, and the microstimulator's third electrode 242c is biased at a second polarity that is opposite to the first polarity. In a third unipolar configuration, the microstimulator's first electrode 242a is held inactive, while the second and third microstimulator electrodes 242b, 242c are oppositely biased at first and second polarities. Any given unipolar configuration can itself correspond to a cathodal or an anodal configuration depending upon the specific polarity of the microstimulator's first and/or second electrode(s) 242a relative to the polarity of the microstimulator's third electrode 242c.

Depending upon embodiment details, switching between bipolar and unipolar configurations, and/or between specific unipolar configurations, can occur in a predetermined, pseudo-random, or aperiodic manner relative to one or more time domains. Such switching can occur in response to a set of commands received from an external programming device 2100 (FIG. 11) and/or another microdevice, or as directed in accordance with an internally-stored set of program instructions. Particular bipolar and/or unipolar stimulation intervals or periods can be identical or different in duration and/or stimulation signal parameters such as current or voltage level, pulse repetition frequency, pulse phase duration(s), burst patterns, or other parameters.

In some embodiments, a remote electrode assembly 460 is implanted beneath a patient's skull, at an anatomical or neurofunctional location that differs from that associated with the first support member 410. Depending upon embodiment details, one or both of the first support member 410 and a remote electrode 460 may be implanted epidurally or subdurally. In other embodiments, a remote electrode assembly 460 is implanted subdermally, for example, beneath a patient's scalp and above the patient's skull.

FIG. 12C is a top schematic view of an MBEA 1104 according to another embodiment of the disclosure. Relative to FIGS. 12A and 12B, like or analogous reference numbers indicate like or analogous elements. In one an embodiment, the MBEA 1104 includes a first and a second remote electrode 460, 462 that are respectively coupled to a microstimulator's first and second electrodes 242a, 242b by first and second remote lead wires 430, 432. The first and second remote lead wires 430, 432 are carried by first and second lead bodies 490, 492 that extend between the first and second remote electrodes 460, 462 and a first support member 410. The microstimulator 206 further includes at least a third electrode 242c that is coupled to one or more contacts 120 carried by the first support member 410.

An MBEA embodiment such as that shown in FIG. 12C can facilitate unipolar signal return or electrical continuity using the first and second remote electrode assemblies 460, 462 simultaneously, or selectively (e.g., in a predetermined, pseudorandom, or aperiodic manner). The remote electrodes 460, 462 can be positioned relative to distinct anatomical regions. For example, the first and second remote electrodes 460, 462 can be positioned epidurally or subdurally relative to different neurofunctional regions in the same brain hemisphere or different brain hemispheres. Alternatively, at least one of the first and second remote electrodes 460, 462 can be positioned subdermally, external to the cranium.

FIG. 13 is a cross section of an implanted MBEA 1100, 1102 implanted in a patient and configured to provide unipolar stimulation according to an embodiment of the disclosure. In this embodiment, the MBEA 1100, 1102 resides beneath a skull plug 96 seated within an opening defined by a craniotomy. Depending upon embodiment details, the remote electrode 460 can be placed in various subdermal locations relative to the skull plug 96, for example, adjacent to or near the skull plug 96; upon or overlapping the skull plug 96; or distant from the skull plug 96. The lengths of the second lead wire 430 and the lead body 490 can differ from one embodiment to another to accommodate a particular type of remote electrode placement.

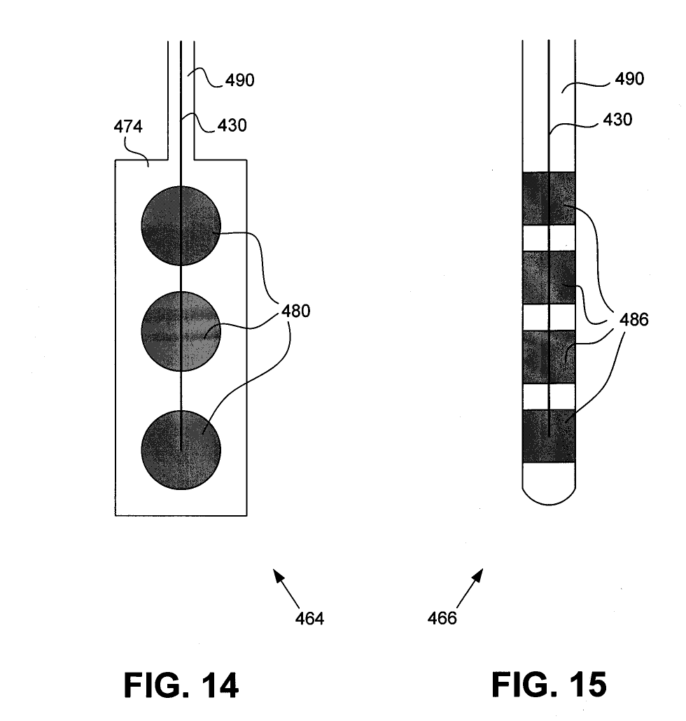

FIGS. 14 and 15 illustrate other embodiments of remote electrodes 464, 466. In one embodiment shown in FIG. 14, a paddle type support member 474 carries a plurality of electrical contacts 480 that are coupled to a remote lead wire 430 that is carried by a lead body 490. In the embodiment shown in FIG. 15, one or more contact bands or segments 486 are carried at particular positions along the length of a lead body 490. The contact segments 486 can extend partially or completely about the circumference or periphery of the lead body 490.

### Microdevice Carried by Lead Body

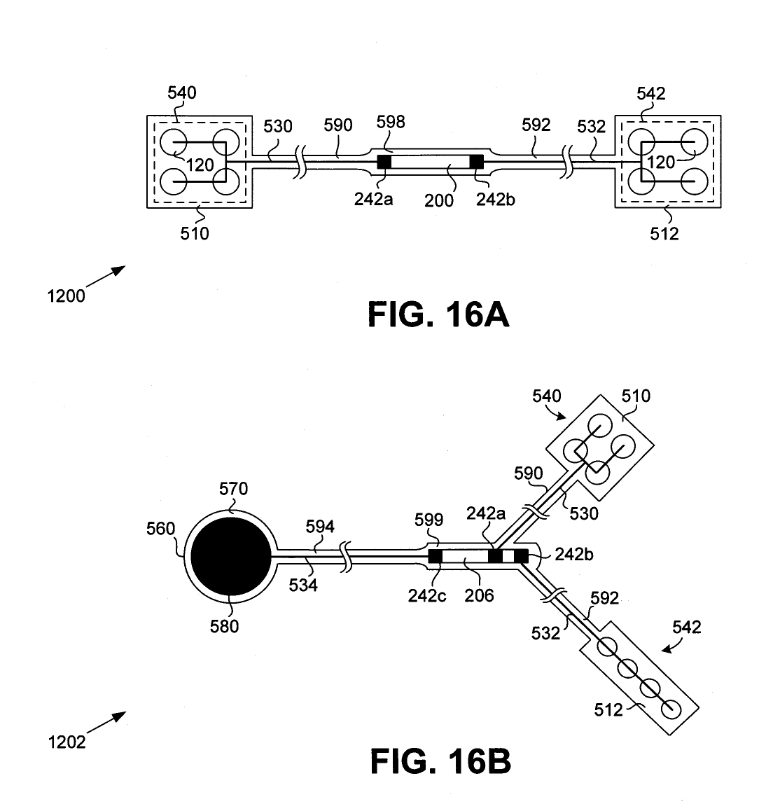

In some embodiments, one or more microstimulators, microsensors, and/or other microdevices can be carried by a portion of a support member that includes a lead body and a set of electrical contacts. FIG. 16A is a schematic illustration of an MBEA 1200 according to an embodiment of the disclosure. In one embodiment, the MBEA 1200 includes a first lead body portion 590, a second lead body portion 592, and a third lead body portion 598. The first and second lead body portions 590, 592 carry lead wires 530, 532 and structurally couple to, extend into, or terminate at a first distal portion 510 and a second distal portion 512, respectively. The first and second distal portions 510, 512 respectively carry a first and a second set of electrical contacts 540, 542. The third lead body portion 598 carries a microstimulator 200. Each contact set 540, 542 is coupled to a corresponding microstimulator electrode 242a, 242b by a corresponding lead wire 530, 532. In certain embodiments, the entire MBEA 1200 can be implanted intracranially. In other embodiments, the first and second support members 510, 512 can be implanted intracranially, while the third lead body portion 598 that carries the microstimulator 200 can be implanted subdermally, external to the cranium. The first and second distal portions 510, 512 can be implanted over the same or different brain hemispheres.

FIG. 16B is a schematic illustration of an MBEA 1202 having a support member according to an embodiment of the disclosure. In one aspect of this embodiment, the MBEA 1202 includes a first, a second, a third, and a fourth lead body portion 590, 592, 594, 599. The first and second lead body portions 590, 592 structurally couple to or transition into a first and a second distal portion 510, 512; and the third lead body portion 594 structurally couples to or transitions into a remote electrode assembly 560, which includes a remote distal portion 570 and a remote electrical contact 580. The fourth lead body portion 599 carries a microstimulator 206, which in the embodiment shown includes a first, a second, and a third electrode 242a, 242b, 242c. The microstimulator's first and second electrodes 242a, 242b are electrically coupled to a first and a second set of electrical contacts 540, 542, which are correspondingly carried by a first and a second distal portions 510, 512. The microstimulator's third electrode 242c is coupled to the remote electrical contact 580 via a lead wire 534.

Depending upon embodiment details and/or clinical application, the first and second distal portions 510, 512 can be implanted intracranially; the fourth lead body portion 599 that carries the microstimulator 206 can be implanted intracranially or extracranially; and the remote electrode assembly 560 can be implanted intracranially or extracranially. In some embodiments, the microstimulator 206 can be selectively or programmably configured to apply or deliver stimulation signals to electrical contacts 120 carried by any two or each of the first distal portion 510, the second distal portion 512, and the remote distal portion 570 at one or more times, in a predetermined, pseudorandom, or aperiodic manner. The first, second, and remote distal portions 510, 512, 570 can be implanted over or in the same or different brain hemispheres. Alternatively, the first distal portion 510 can be implanted relative to a brain location, the second distal portion 512 can be implanted relative to a spinal column or peripheral nervous system (e.g., cranial nerve) location; and the remote support member 570 can be implanted at another anatomical location (e.g., beneath the scalp and above the skull; or in a subclavicular location).

In some embodiments, the MBEAs 1200, 1202 of FIGS. 16A and 16B include one or more microsensors (e.g., such as those described herein) in addition to or instead of microstimulators. In embodiments in which an MBEA 1200, 1202 includes a microsensor, the microsensor can be carried by a portion of a lead body (e.g., proximate to or remote from a portion of the lead body that carries a microstimulator); or the microsensor can be carried by a support member to which a portion of a lead body is structurally coupled.

### Embodiments Including Fluid Transfer Devices

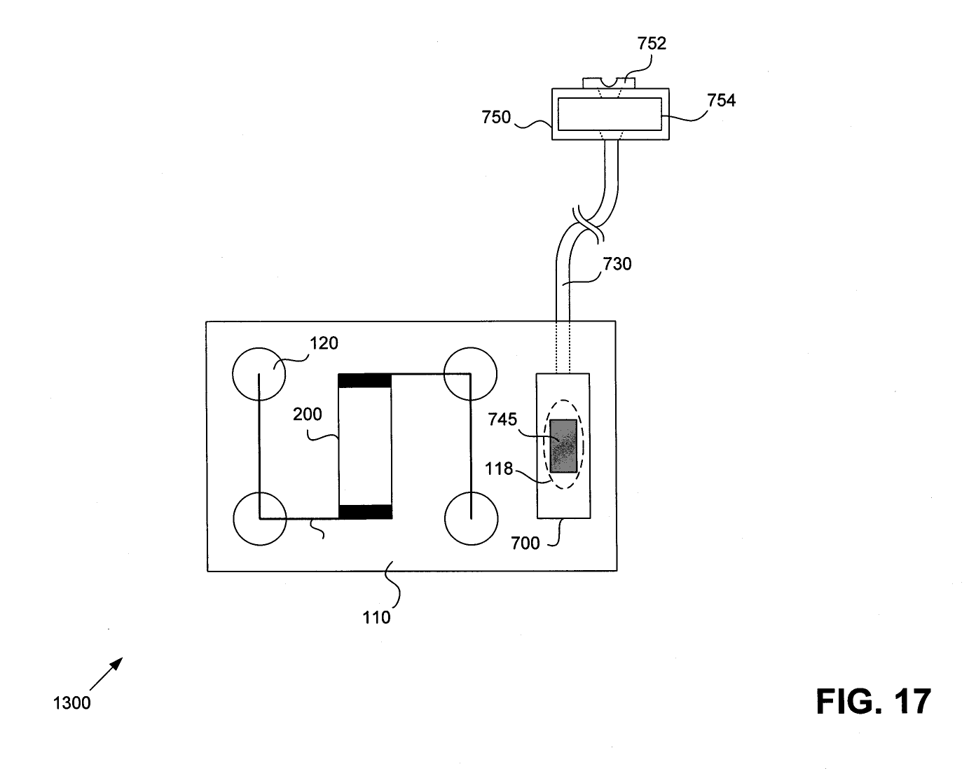

Certain embodiments in accordance with the present disclosure can include microinfusion, microextraction, and/or other types of fluid transfer devices. Such devices can be carried by various portions of support members and/or lead bodies. FIG. 17 is a schematic illustration of a microstimulation and microfluidic assembly 1300 in accordance with an embodiment of the disclosure. In one embodiment, the assembly 1300 includes a support member 110 that carries at least one microfluidic delivery and/or extraction device 700; and at least microstimulator 200 coupled to a set of electrical contacts 120. The microfluidic delivery and/or extraction device 700 is structurally coupled to a reservoir structure 750 by at least one fluid transfer line or catheter 730, which facilitates the flow of fluid(s) between the reservoir structure 750 and the microfluidic delivery and/or extraction device 700. The fluid transfer line 730 extends into or is embedded into a portion of the support member 110 to facilitate structural coupling to the microfluidic delivery and/or extraction device. The fluid transfer line 730 can be, for example, a Silicone-based or other type of biocompatible material having an opening or channel through which fluid can flow.

The microstimulator 200 is configured to apply one or more types of neural stimulation (e.g., bipolar or unipolar stimulation signals) to a target neural population at one or more times, in a manner analogous to that described above for various MBEA embodiments. The assembly 1300 can include additional support member structures, for example, the assembly 1300 can include or comprises an MBEA having a remote electrode structure that facilitates the application of unipolar stimulation signals. In some embodiments, the assembly 1300 can include one or more microsensors 300 instead of or in addition to one or more microstimulators 200. Additionally or alternatively, one or more mini-pumps or micropumps can be carried by or reside within the reservoir structure 750 instead of or in addition to particular microfluidic delivery and/or extraction devices 700 that are carried by the support member 110. A reservoir-resident pump can be structurally coupled to a fluid transfer line 730 that extends or is embedded into a portion of the support member 110 (e.g., adjacent to or proximate to particular electrical contacts 120, terminating at an aperture or window 118 in the support member 110).

Depending upon embodiment details, the microfluidic delivery and/or extraction device 700 can deliver or apply, and/or withdraw or extract, substances or fluids from an anatomical location at, above, adjacent, or proximate to a substance delivery or extraction site relative to which the assembly 1300 is implanted. The microfluidic delivery and/or extraction device 700 includes a set of fluid transfer openings or interfaces 745 that facilitate fluid delivery and/or extraction. In a representative embodiment, the microfluidic delivery and/or extraction device can be identical, analogous, or similar to a device described in U.S. Pat. No. 6,733,485. In several embodiments, the support member 110 includes an aperture or window 118 that facilitates such fluid transfer operation(s).

The reservoir structure 750 includes a housing and one or more compartments, receptacles, or chambers 754 for storing or receiving one or more types of fluids. The reservoir structure further includes a set of filling and/or withdrawal ports 752. Depending upon embodiment details, the reservoir structure 750 can include a set of microvalves and/or other devices that facilitate fluid transfer in a particular direction. In some embodiments, the reservoir structure 750 can be implanted subdermally, within a recess in the skull, in a manner that facilitates identification or localization (for example, in association with a palpation process, as understood by those of ordinary skill in the relevant art (e.g., medical professionals experienced in filling an intrathecal Baclofen pump via an injection process)) of the filling and/or withdrawal port(s) 752.

The support member 110 can be implanted relative to one or more brain regions, such that the microstimulator 200 can apply stimulation signals to particular target neural populations, and the fluid delivery and/or extraction device 700 can transfer substances to and/or from adjacent or proximate neural or other tissue. The fluid delivery and/or extraction device 700 can introduce one or more substances (e.g., a growth factor) to or in the vicinity of a target neural population, for example, to facilitate a given therapeutic outcome such as functional development or recovery (e.g., as a result of neuroplastic processes). Such a therapeutic outcome can be facilitated or enhanced by neural stimulation (for example, applied at a subthreshold level that is approximately 25%-75% of a patient response threshold (e.g., a motor, sensation, cognitive, or other threshold), using a pulse repetition frequency of approximately 20 Hz-120 Hz (e.g., 50 Hz, 75 Hz, or 100 Hz), a first phase pulse width of approximately 100 microseconds-250 microseconds, and a unipolar (e.g., cathodal or anodal) and/or bipolar polarity) at one or more times. The neural stimulation can be applied in association with or during one or more portions of a behavioral therapy session (e.g., physical therapy, speech therapy, or cognitive therapy) to further enhance neurofunctional gains.

In some embodiments, the fluid delivery and/or extraction device 700 can periodically (e.g., one or more times per week or month, or after about 3-6 months) extract or withdraw fluid (e.g., CSF) and transfer the withdrawn fluid to the reservoir structure 750. Such fluid can be extracted for subsequent analysis (e.g., to identify biological markers (for example, associated with cellular metabolism or neuroplasticity)) to determine whether a neural stimulation procedure is having an intended (or unintended) effect. Based upon such an analysis, one or more neural stimulation parameters (e.g., a current or voltage level, pulse repetition frequency, pulse phase width, pulse polarity, signal application location, and/or stimulation period duration) can be maintained or adjusted, or a neural stimulation procedure can be temporarily interrupted or terminated.

In certain embodiments, at one or more times a fluid delivery and/or extraction device 700 can introduce or apply non-fluidic, partially fluidic, or quasi-fluidic substances or materials that can be carried by or within a fluid to particular target tissues. For example, a fluid that contains particular types of nanostructures or nanodevices (e.g., gold nanospheres) can be introduced into the reservoir structure, and fluid delivery and/or extraction device 700 can subsequently apply or deliver such nanostructures or nanodevices to a target location within the body. As another example, a fluid that contains or carries particular types of biological structures, such as partially or fully undifferentiated cells (e.g., stem cells or precursor cells), can be delivered to a target location within the body in an analogous or similar manner.

One feature of at least some of the foregoing embodiments is that they can include microstimulators and/or other microdevices that are carried by support members that in turn include signal delivery electrodes and/or other patient interfaces. One advantage of this arrangement is that it can allow a manufacturer to use the signal generation capability of a prepackaged, small signal generator to provide a product with a significantly wider signal distribution than is available with the signal generator alone, without tampering with, damaging, and/or impinging on the integrity of the housing in which the signal generator is contained. For example, the support member can be attached to an external surface of the housing, and need not penetrate into the interior region of the housing. In another aspect of this example, the structural connection between the housing and the support member is at a different location then the electrical connection between microstimulator electrodes and the support member electrodes. This arrangement can facilitate the ability of the support member to carry the microstimulator.

Another advantage of particular embodiments of the foregoing systems is that they can include a switch that allows the practitioner (and optionally, the patient) to select different target areas of the patient for treatment. Accordingly, a single device can be used to treat multiple target sites and/or multiple disfunctions.

Still another advantage of at least some of the foregoing embodiments is that the support member can provide stability to the microdevice. Accordingly, the microdevice can be less likely to become dislodged from its initially implanted location and can accordingly be more likely to sustain a planned treatment regimen over the course of time.

From the foregoing, it will be appreciated that specific embodiments of the disclosure have been described herein for purposes of illustration, but that various modifications may be made without deviating from the invention. For example, the support member can surround or partially surround the microdevice it carries by techniques other than “sandwiching,” e.g., by injection molding, overmolding, encapsulating, or other suitable methods. In another example, when the microdevice carried by the support member provides or receives electrical signals, it can include electrical terminals to which an electrical signal path is connected. When the microdevice provides or receives fluids or fluid signals, it can include fluid terminals. In other embodiments, the microdevice can include other types of terminals. As a result, microdevices in accordance with several embodiments of the disclosure can send fluids or signals to the patient, and/or receive signals or fluids from the patient via an appropriately selected signal/fluid transmitter/receiver, and an appropriately selected interface. For example, when the microdevice includes a microstimulator, the system includes a pulse generator, and the interface includes an electrical contact or electrode. When the microdevice includes a fluid infusion device, the system can include a pump, and the interface can include a delivery tube. When the microdevice includes a fluid extraction device, the system can include a pump or vacuum device, and the interface can include an extraction tube. A microdevice that includes a sensor can include an interface with an appropriate sensor probe.

Certain aspects of the invention described in the context of particular embodiments may be combined or eliminated in other embodiments. For example, characteristics of the systems described in the context of electrical microstimulators can be applied as well to other microdevices, e.g., microsensors, microinfusers and/or microextractors. Microdevices or microdevice combinations shown in certain Figures may be combined with features of other Figures. Further, while advantages associated with certain embodiments have been described in the context of those embodiments, other embodiments may also exhibit such advantages, and not all embodiments need necessarily exhibit such advantages. Accordingly, the disclosure can include other embodiments not shown or described above.

I/We claim:

1. A patient treatment system, comprising:
a patient-implantable, flexible support member having a first and second edge;
a plurality of 1×3 array of electrodes with a first of the plurality of 1×3 array of electrodes carried by the support member at the first edge of the support member and a second of the plurality of 1×3 array of electrodes carried by the support member at the second edge of the support member; and
a microstimulator including a housing, a pulse generator internal to the housing, the microstimulator carried by the support member intermediate the first of the plurality of 1×3 array of electrodes and the second of the plurality of 1×3 array of electrodes, the microstimulator being electrically connected to each of the plurality of 1×3 array of electrodes.

2. The system of claim 1, wherein the microstimulator is positioned generally parallel to at least one of the plurality of 1×3 array of electrodes.

3. The system of claim 1, wherein the microstimulator is positioned generally perpendicular to at least one of the plurality of 1×3 array of electrodes.

4. The system of claim 1, wherein for at least one of the plurality of 1×3 array of electrodes each of the electrodes are electrically connected together.

5. The system of claim 1, wherein the microstimulator includes a first end and a second end, and includes an electrode positioned at each of the first end and the second end.

6. The system of claim 5, and further including at least one lead wire, the at least one lead wire electrically connecting the electrode at the first end of the microstimulator with at least one of the electrodes of the first of the plurality of 1×3 array of electrodes, the lead wire further for reducing mechanical stress.

7. A patient treatment system, comprising:
a patient-implantable, flexible support member having a first and second edge;
a first and second 1×3 array of electrodes with the first array of electrodes carried by the support member at the first edge of the support member and a second array of electrodes carried by the support member at the second edge of the support member; and
an elongated microstimulator including a housing, a pulse generator internal to the housing, the microstimulator carried by the support member intermediate the first array of electrodes and the second array of electrodes, the microstimulator being electrically connected to the first array of electrodes and the second array of electrodes.

8. The system of claim 7, including a first lead wire and a second lead wire, with the first lead wire electrically connecting the microstimulator with at least one of the electrodes of the first array of electrodes, the second lead wire electrically connecting the microstimulator with at least one of the electrodes of the second array of electrodes.

9. The system of claim 8, where each of the first and second lead wires further for reducing mechanical stress.

10. The system of claim 9, wherein the microstimulator includes a first end and a second end, and includes an electrode positioned at each of the first end and the second end.

11. The system of claim 10, wherein the first lead wire electrically connects the first electrode of the microstimulator with the at least one of the electrodes of the first array of electrodes.

12. The system of claim 11, wherein the second lead wire electrically connects the second electrode of the microstimulator with the at least one of the electrodes of the second array of electrodes.

13. The system of claim 12, wherein the microstimulator is positioned generally parallel to at least one of the first and second arrays of electrodes.

14. The system of claim 12 wherein the microstimulator is positioned generally perpendicular to at least one of the first and second arrays of electrodes.

15. The system of claim 12, wherein each of the electrodes for at least one of the first and second arrays of electrodes are electrically connected together.
